(19) United States
(12) Patent Application Publication
(10) Pub. No.: US 2023/0321325 A1
(43) Pub. Date: Oct. 12, 2023

(54) BIOABSORBABLE FLOW DIVERTING SCAFFOLD AND METHODS FOR ITS USE

(71) Applicant: Fluid Biomed Inc., Calgary (CA)

(72) Inventors: Alim P. Mitha, Calgary (CA); John H. Wong, Calgary (CA)

(73) Assignee: Fluid Biomed Inc., Calgary (CA)

(21) Appl. No.: 18/336,241

(22) Filed: Jun. 16, 2023

Related U.S. Application Data
(63) Continuation of application No. 17/738,277, filed on May 6, 2022.
Application No. 17/738,277 is a continuation-in-part of application No. 16/810,679, filed on Mar. 5, 2020.
Application No. 16/810,679 is a continuation of application No. PCT/CA2019/050304, filed on Mar. 12, 2019.
(60) Provisional application No. 62/641,891, filed on Mar. 12, 2018.

(51) Int. Cl. A61L 27/58; A61L 27/56; A61L 27/06; A61L 27/04; A61L 27/18; A61F 2/90; A61F 2/07

(52) U.S. Cl. CPC A61L 27/58; A61L 27/56; A61L 27/06; A61L 27/045; A61L 27/18; A61F 2/90; A61F 2/07; A61F 2310/00023; A61F 2210/0004; A61F 2250/0098; A61F 2310/00029

(57) ABSTRACT

This disclosure relates to scaffolds made of a braid of bioabsorbable polymeric fibers for implantation within a lumen of a mammalian body and, in particular, to such scaffolds that are configured to divert blood flow from a pathology associated with a blood vessel.

### CROSS-REFERENCE TO RELATED APPLICATION

The present application is a continuation of U.S. patent application Ser. No. 17/738,277 (Attorney Docket No. 62197-703.501), filed May 6, 2022, which is a continuation-in-part of U.S. application Ser. No. 16/810,679 (Attorney Docket No. 62197-703.301), filed on Mar. 5, 2020, which is a continuation of PCT/CA2019/050304 (Attorney Docket No. 62197-703.601), filed on Mar. 12, 2019, which claims the benefit of U.S. provisional application 62/641,891 (Attorney Docket No. 62197-703.101), filed on Mar. 12, 2018, the full disclosures of which are incorporated herein by reference.

### BACKGROUND OF THE INVENTION

1. Field of the Invention. This disclosure relates to scaffolds made of a braid of bioabsorbable polymeric fibers for implantation within a lumen of a mammalian body. Particular aspects of this disclosure relate scaffolds made from such braids that are configured to divert blood flow from a pathology associated with a blood vessel.

2. Description of the Background Art. There is an abundance of medical devices that are known in the art, which are implanted into blood vessels in the body to treat various pathologies. For example, an aneurysm is an outward bulging, balloon-like structure caused by a localized weak spot on a blood vessel wall. Aneurysms have thin, weak walls and are thus at risk of rupturing. “Flow-diverting” scaffolds have been proposed to treat aneurysms, by which a stent is inserted to span the neck of an aneurysm in order to divert flow past the aneurysm and thus allow it to heal. Flow diversion thus removes the need to enter an aneurysm. Such flow-diverting scaffolds are described in, for example, U.S. Pat. Nos. 8,715,312 and 8,267,986. U.S. Pat. Nos. 8,715,312 and 8,267,986 describe scaffolds made of braided metal wires. The Pipeline™ Flex embolization device (Medtronic) is used for endovascular treatment of large or giant wide-necked intracranial aneurysms. The Pipeline™ Flex device consists of 75% cobalt chromium/25% platinum tungsten wires.

The metal composition of the flow-diverting scaffolds known in the art provides disadvantages. As they are permanent and cannot be removed, they present various drawbacks, including risk of thrombosis that requires patients to remain anti-platelet medications long-term, risk of hyperplasia, prevention of vascular lumen remodeling or expansion, and occlusion of the blood vessel. Metal scaffolds are also present disadvantages in the context of CT and MRI imaging post-implantation as the signal they reflect tends to be too bright.

Accordingly, there is a need for implantable devices that eliminate or reduce the negative responses of the body at the implantation site while allowing for the prevention or treatment of a disease. Bioresorbable scaffolds have advantages compared to the metal scaffolds, including non-permanency. However, clinical studies showed that bioabsorbable coronary have higher risks of thrombosis (Masayuki et al., Circulation 136, A15796-A15796; Raber et al., ACC (Journal Am. Coll. Cardiol. 66, 1901-1914; Kang et al., ACC Cardiovasc. Interv. 9, 1203-1212). Furthermore, Waksmen et al. (Circ. Cardiovasc. Interv. 10, e004762) have demonstrated the higher thrombogenicity of scaffolds made with the bioabsorbable polymer nature of PLLA.

### SUMMARY OF THE INVENTION

Other aspects and features of the present invention will become apparent to those ordinarily skilled in the art upon review of the following description of specific embodiments of the invention in conjunction with the accompanying figures.

Aspects of the disclosure relate to a device comprising a resiliently deformable tubular body for positioning in a body lumen defined by a body wall, the tubular body comprising a braid of interwoven bioabsorbable polymeric fibers, wherein the tubular body comprises at least 38 polymeric fibers. In various embodiments, when the device is in an expanded formation, the braid has a porosity in the range of about 5% to about 80%. In various embodiments, when the device is in an expanded formation, the braid has a porosity in the range of about 60% to about 80%.

Aspects of the disclosure relate to a device comprising a resiliently deformable tubular body for positioning in a body lumen defined by a body wall, the tubular body comprising a braid of interwoven bioabsorbable polymeric fibers, wherein, when the device is in an expanded formation, the braid has a porosity in the range of about 60% to about 80%. In various embodiments, the tubular body comprises at least 38 polymeric fibers.

In various embodiments of the devices described above, the braid comprises 38 to 96 bioabsorbable polymeric fibers. In various embodiments, the braid comprises at least 44 bioabsorbable polymeric fibers. In various embodiments, the braid comprises at least 46 bioabsorbable polymeric fibers. In various embodiments, the braid comprises at least 48 bioabsorbable polymeric fibers. In various embodiments, the braid comprises at least 72 bioabsorbable polymeric fibers. In various embodiments, the braid comprises 44 bioabsorbable polymeric fibers. In various embodiments, the braid comprises 46 bioabsorbable polymeric fibers. In various embodiments, the braid comprises 48 bioabsorbable polymeric fibers. In various embodiments, wherein the braid comprises 72 bioabsorbable polymeric fibers. In various embodiments, the braid comprises at least 96 bioabsorbable polymeric fibers.

In various embodiments of the devices described above, the bioabsorbable polymeric fibers have a diameter of at least about 30 μm. In various embodiments, the bioabsorbable polymeric fibers have a diameter in the range of about 30 μm to about 80 μm. In various embodiments, the bioabsorbable polymeric fibers have a diameter of about 40 μm. In various embodiments, bioabsorbable polymeric fibers have a diameter of about 50 μm. In various embodiments, the bioabsorbable have a diameter of about 60 μm. In various embodiments, the bioabsorbable polymeric fibers have a diameter of about 70 μm. In various embodiments, the bioabsorbable polymeric fibers have a diameter of about 80 μm.

In various embodiments of the devices described above, the bioabsorbable polymeric fibers are interwoven in a 2-under-2-over-2 pattern. In various embodiments, the bioabsorbable polymeric fibers are interwoven in a 1-over-2-under-2 pattern. In various embodiments, the bioabsorbable polymeric fibers are interwoven in 1-over-1-under-1 pattern.

In various embodiments of the devices described above, the diameter of the tubular body is about 4 mm. In various embodiments, the bioabsorbable polymeric fibers are interwoven at a pitch angle of about 16° or less. In various embodiments, the bioabsorbable polymeric fibers are interwoven at a pitch angle of about 14° or less.

In various embodiments of the devices described above, the diameter of the tubular body is about 5 mm. In various embodiments, the bioabsorbable polymeric fibers are interwoven at a pitch angle of about 12, or less. In various embodiments, the bioabsorbable polymeric fibers are interwoven at a pitch angle of about 10° or less.

In various embodiments of the devices described above, the diameter of the tubular body is about 3 mm. In various embodiments, the bioabsorbable polymeric fibers are interwoven at a pitch angle of about 18° or less. In various embodiments, the bioabsorbable polymeric fibers are interwoven at a pitch angle of about 16° or less.

In various embodiments of the devices described above, the diameter of the tubular body is about 7 mm. In various embodiments, the bioabsorbable polymeric fibers are interwoven at a pitch angle of about 9° or less.

In various embodiments of the devices described above, when the device is in an expanded formation the braid has a pore density of in the range of about 10 pores/mm² to about 32 pores/mm².

In various embodiments of the devices described above, the tubular body further comprises a visualization aid. In various embodiments, the visualization aid comprises a radio-opaque material. In various embodiments, the radio opaque material comprises iodine or barium. In various embodiments, the visualization aid comprises at least one wire comprising a radio-opaque material, wherein each wire is interwoven with the plurality of bioabsorbable polymeric fibers to form part of the braid.

In various embodiments of the devices described above, the tubular body comprises means for facilitating and/or maintaining radial and/or axial expansion of the tubular body in the body lumen. In various embodiments, the means for facilitating and maintaining expansion of the tubular body in the body lumen is at least one wire, wherein each wire is interwoven with the plurality of bioabsorbable polymeric fibers to form part of the braid. In various embodiments, the at least one wire comprises a radio opaque material.

In various embodiments of the devices described above, the at least one wire is a resiliently deformable wire. In various embodiments, the resiliently deformable wire comprises a nickel-titanium alloy or a cobalt-chromium-nickel alloy. In various embodiments, each wire independently comprises: a nickel-titanium alloy coated with the radio-opaque material; a drawn filled tube (DFT) comprising a nickel-titanium alloy exterior and a core comprising the radio-opaque material; a DFT comprising an exterior comprising the radio-opaque material and a core comprising a nickel-titanium alloy; a cobalt-chromium-nickel alloy coated with the radio-opaque material; a (DFT) comprising a cobalt-chromium-nickel alloy exterior and a core comprising the radio-opaque material; or a DFT comprising an exterior comprising the radio-opaque material and a core comprising cobalt-chromium-nickel alloy.

In various embodiments of the devices described above, the radio-opaque material comprises iodine or barium.

In various embodiments of the devices described above, the radio-opaque material comprises a radio-opaque metal. In various embodiments, the radio-opaque metal is tantalum, gold, platinum, or a combination thereof.

In various embodiments of the devices described above, the at least one wire comprises a tantalum-coated nitinol wire.

In various embodiments of the devices described above, the at least one wire comprises a DFT comprising a nitinol exterior and a platinum core.

In various embodiments of the devices described above, the at least one wire comprises 2 wires, 3 wires, 4 wires, 5 wires, 6 wires, 7 wires, 8 wires, 9 wires, or 10 wires.

In various embodiments of the devices described above, the plurality of polymeric fibers comprise polylactides (PLA), polyglycolides (PGA), polycaprolactone (PCL), polylactide-co-glycolides (PLGA), polyanhydrides, polyorthoesters, poly(N-(2-hydroxypropyl) methacrylamide), poly(l-aspartamide), DLPLA-poly(dl-lactide), poly (L-Lactic acid); LPLA-poly(l-lactide), PDO-poly (dioxanone), PGA-TMC-poly (polyglycolide-co-trimethylene carbonate), PGA-LPLA-poly(l-lactide-co-glycolide), PGA-DLPLA-poly(dl-lactide-co-glycolide), LPLA-DLPLA-poly(l-lactide-co-dl-lactide), PDO-PGA-TMC-poly(glycolide-co-trimethylene carbonate-co-dioxanone), or any combination thereof. In various embodiments, the plurality of polymeric fiber comprise polylactides (PLA), polylactide-co-glycolides (PLGA), DLPLA-poly(dl-lactide), poly-L-Lactic acid), LPLA-poly(l-lactide), PGA-LPLA-poly(l-lactide-co-glycolide), PGA-DLPLA-poly(dl-lactide-co-glycolide), LPLA-DLPLA-poly(l-lactide-co-dl-lactide), or any combination thereof. In various embodiments, the plurality of polymeric fibers comprise poly-L-lactic acid (PLLA).

In various embodiments of the devices described above, the tubular body comprises a therapeutic agent conjugated to the bioabsorbable polymeric fibers. In various embodiments, the bioabsorbable polymeric fibers are coated with a therapeutic agent. In various embodiments, the therapeutic agent is an antibiotic agent, an antiviral agent, an analgesic, a muscle relaxant, a chemotherapeutic agent, an intra-arterial vasodilating agent, a calcium channel inhibitor, a calcium channel antagonist, a calcium channel blocker, a transient receptor potential protein blocker, an endothelin antagonist, a blood thinning agent, an antiplatelet agent, or any combination thereof. In various embodiments, the therapeutic agent is aspirin, heparin, Ticagrelor, 5-fluorouracil, melphalan, or clopidogrel. In various embodiments, the therapeutic agent is paclitaxel, sirolimus, everolimus, temozolamide, cyclophosphamide, doxorubicin, irinotecan, azathioprine, methotrexate, cisplatin, or vincristine.

In various embodiments of the devices described above, the lumen is the lumen of a blood vessel. In various embodiments, the blood vessel is an intracranial vessel. In various embodiments, the device is for positioning adjacent to a pathology of the blood vessel to divert blood flow from the pathology. In various embodiments, the pathology is an aneurysm, a cancer, an infection, coronary artery disease, carotid artery atherosclerotic disease, or intracranial atherosclerosis.

In various embodiments of the devices described above, the device is for positioning in the lumen at a site adjacent to a pathology of or proximal to the body wall to supply lactic acid to the site.

Aspects of the disclosure relate to use of a device as defined above for deployment within a lumen of a body to treat a pathology of or proximal to a body wall defining the lumen. Aspects of the disclosure relate to use of a device as defined above for deployment within a body lumen to deliver a therapeutic agent to a pathology of or proximal to a body wall defining the lumen. Aspects of the disclosure relate to use of a device as defined above for deployment within a body lumen to deliver lactic acid to a site proximal to a pathology of or proximal to a body wall defining the lumen. In various embodiments, the body wall is the wall of a blood vessel. In various embodiments, the blood vessel is an intracranial blood vessel. In various embodiments, the pathology is an aneurysm, a cancer, an infection, coronary artery disease, carotid artery atherosclerotic disease, or intracranial atherosclerosis.

Aspects of the disclosure relate to a method of treating a pathology of or proximal to a body wall, the method comprising deploying a device as defined in above in a lumen defined by the body wall at a position proximal to the pathology. Aspects of the disclosure relate to a method of delivering lactic acid to a site proximal to a pathology of, or proximal to, a body wall, the method comprising deploying a device as described above within a lumen defined by the body wall at the site proximal to the pathology. In various embodiments, the body wall is the wall of a blood vessel. In various embodiments, the blood vessel is an intracranial blood vessel. In various embodiments, the pathology is an aneurysm, a cancer, an infection, coronary artery disease, carotid artery atherosclerotic disease, or intracranial atherosclerosis

In a further aspect, the present invention provides a method for diverting blood flow in an intracranial blood vessel away from an aneurysm. The method comprises expanding a device comprising a resiliently deformable porous tubular body in a lumen of the intracranial blood vessel across a neck of the aneurysm, where the resiliently deformable porous tubular body comprises metal wires interwoven with bioabsorbable polymeric fibers to form a braid. The metal wires are resiliently deformable and configured to fully expand the tubular structure against the body wall, and the braid has a porosity when in an expanded configuration selected to permit a small amount of blood to enter the aneurysm with low velocity which causes thrombosis and occlusion of the aneurysm and permits the aneurysm to heal. The bioabsorbable polymeric fibers degrade over time leaving only the metal wires in place.

In preferred instances, the braid has a porosity in a range of about 60% to about 80% when the tubular body is expanded.

In preferred instances, the braid comprises (1) at least 38 bioabsorbable polymeric fibers and (2) from 2 to 12 resiliently deformable interwoven metal wires.

In preferred instances, the bioabsorbable polymeric fibers have a diameter in the range of about 30 μm to about 80 μm.

In preferred instances, the metal wires are radiopaque and the method further comprises visualizing the radiopaque wires while the tubular body of the device is being deployed.

In preferred instances, the resiliently deformable wire comprises a nickel-titanium alloy or a cobalt-chromium-nickel alloy.

In preferred instances, the bioabsorbable polymeric fibers comprise one or more materials selected from the group consisting of polylactides (PLA), polylactide-co-glycolides (PLGA), DLPLA-poly(dl-lactide), poly-L-Lactic acid), LPLA-poly(l-lactide), PGA-LPLA-poly(l-lactide-co-glycolide), PGA-DLPLA-poly(dl-lactide-co-glycolide), LPLA-DLPLA-poly(l-lactide-co-dl-lactide), or any combination thereof.

In preferred instances, the methods further comprise delivering an occlusive material to the aneurysm after the porous tubular body has been expanded across the neck of the aneurysm. For example, the occlusive material may be delivered to the aneurysm after the porous tubular body has been expanded across the neck of the aneurysm comprises advancing a distal tip of a microcatheter through the braid and delivering the occlusive material through the microcatheter, e.g., the microcatheter may be advanced through the polymeric fibers causing the braid to expand over the microcatheter when introduced and to collapse when the microcatheter is withdrawn. Typically, the occlusive material comprises occluding coils.

In specific instances, the metal wires are formed from a metal have a modulus of elasticity in a range from 5 GPa to 30 GPa and the bioabsorbable polymeric fibers are formed from a polymer have a modulus of elasticity in a range from 2 GPa to 10 GPa. For example, the metal wires may be formed from a nickel-titanium alloy and the bioabsorbable polymeric fibers are formed from PLLA.

In specific instances, the resiliently deformable porous tubular body may be shaped across and/or into at least the neck of the aneurysm.

In specific instances, the resiliently deformable porous tubular body may be expanded across a neck of a sidewall aneurysm.

In specific instances, the resiliently deformable porous tubular body may be expanded across and/or into (a) the neck of a bifurcated aneurysm and/or (b) the neck of a branch lumen.

In a still further aspect, the present invention provides method for diverting blood flow in an intracranial blood vessel away from an aneurysm located at a bifurcation. The method comprises releasing and expanding a device comprising a resiliently deformable porous tubular body from a lumen of the intracranial blood vessel into one of the two branch lumens, where the resiliently deformable porous tubular body comprises metal wires interwoven with bioabsorbable polymeric fibers to form a braid. Typically, the metal wires are resiliently deformable and configured to fully expand the tubular structure against the body wall. The resiliently deformable porous tubular body may be “shaped” to cross and/or protrude into at least one of (a) a neck of the aneurysm and (b) the other of the two branch lumens after the resiliently deformable porous tubular body has been expanded. The braid typically has a porosity when in an expanded configuration selected to permit a small amount of blood to enter the aneurysm with low velocity which causes thrombosis and occlusion of the aneurysm and permits the aneurysm to heal, and the bioabsorbable polymeric fibers degrade over time leaving only the metal wires in place.

In specific instances, the resiliently deformable porous tubular body may be shaped to cross and/or protrude into at least the neck of the aneurysm.

In specific instances, the resiliently deformable porous tubular body may be shaped to cross and/or protrude into at least the other of the two branch lumens.

In preferred instances, the resiliently deformable porous tubular body may be shaped to cross and/or protrude into both (a) the neck of the aneurysm and (b) the other of the two branch lumens.

In preferred instances, expanding the device may comprise releasing the device from constraint within a microcatheter and shaping the resiliently deformable porous tubular body may comprise pushing on the resiliently deformable porous tubular body with the microcatheter and/or a pusher member advanced through the microcatheter.

In specific instances, the metal wires may be formed from a metal have a modulus of elasticity in a range from 5 GPa to 30 GPa and the bioabsorbable polymeric fibers are formed from a polymer have a modulus of elasticity in a range from 2 GPa to 10 GPa.

In preferred instances, the metal wires may be formed from a nickel-titanium alloy and the bioabsorbable polymeric fibers are formed from PLLA (poly L-lactic acid).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an isometric view of an implantable device comprising a braid of interwoven bioabsorbable polymeric fibers according to a first embodiment;

FIG. 2 is a picture of an embodiment of an implantable endovascular device comprising 48 interwoven poly L-lactic acid (PLLA) polymeric fibers;

FIG. 3 is a picture of an embodiment of an implantable endovascular device comprising 48 interwoven poly L-lactic acid (PLLA) polymeric fibers showing the resilient deformability of the device;

FIG. 4 is a schematic diagram of an implantable device comprising a braid of interwoven fibers that illustrates pitch angle.

FIG. 5 is a schematic diagram of a braiding machine useful for manufacturing devices of the present disclosure.

FIG. 6A is an isometric view of an implantable device according to a second embodiment of the invention comprising a braid of interwoven bioabsorbable polymeric fibers and radio-opaque material;

FIG. 6B is a side view of the device illustrated in FIG. 6A;

FIG. 7A is a picture of an embodiment of an implantable endovascular device comprising 44 interwoven poly L-lactic acid (PLLA) polymeric fibers and 4 radio-opaque wires;

FIG. 7B is a close up picture of the device of FIG. 7A;

FIG. 8A is a picture of an embodiment of an implantable endovascular device comprising 46 interwoven poly L-lactic acid (PLLA) polymeric fibers and 2 radio-opaque wires;

FIG. 8B is a close up picture of the device of FIG. 8A;

FIG. 9A is a schematic diagram of a flow diverting application to treat of an aneurysm;

FIG. 9B is a schematic diagram of a flow diverting application to treat of an aneurysm;

FIG. 10 is a schematic diagram of a flow diverting application in combination with an aneurysm-bridging application;

FIG. 11A is an early arterial phase angiogram taken before device implantation, showing an aneurysm created in a rabbit carotid artery with a daughter sac at the tip of the aneurysm;

FIG. 11B is an early venous phase angiogram of the same aneurysm shown in FIG. 19A (same angiographic run as above) before device implantation, demonstrating rapid contrast washout except in the daughter sac;

FIG. 11C is an early venous phase angiogram of the same aneurysm shown in FIGS. 19A and 19B after placement of the device, demonstrating contrast stagnation in the body of the aneurysm indicative of a flow diverting effect;

FIG. 12A is an angiogram of a rabbit aorta immediately after implantation of a device comprising 44 bioabsorbable PLA fibers and radio-opaque Tantalum-coated nitinol fibers;

FIG. 12B is an angiogram of the rabbit aorta depicted in FIG. 14A 1 month after implantation of the device;

FIG. 13 is a scanning electron micrograph (SEM) showing persistent patency of a side branch of a rabbit aorta 1 month after implantation of the device;

FIG. 14 is a gross histology picture of a device comprising 44 bioabsorbable PLA fibers and 4 radio-opaque Tantalum-coated nitinol fibers after implantation in a rabbit aorta;

FIG. 15 are scanning electron micrographs (SEM) showing a smooth neointimal layer forming over the stent struts 1 month after implantation of the device into the rabbit aorta;

FIG. 16A is a histological cross section of a rabbit aorta showing persistence of polymer fibers and neointima formation over the fibers one month after implantation of a device;

FIG. 16B is a histological cross section of a rabbit aorta showing persistence of polymer fibers, neointima formation over the fibers, and a lack of exuberant inflammatory response two months after implantation of a device.

FIG. 17 is a picture of a device according to an embodiment disclosed herein consisting only of bioabsorbable PLLA polymeric fibers, illustrating its ability to self-expand after being loaded into, then pushed out of, a catheter with an inner diameter of 0.027″.

FIGS. 18A-18F illustrate the delivery of a flow diverting stent to a cerebral aneurysm located at a vascular bifurcation in accordance with the principles of the present invention.

FIG. 19 illustrates the delivery of occluding coils through the flow diverting stent of FIGS. 18A-18F.

### DEFINITIONS

“Pathology” as used herein refers to the structural and functional deviations from the normal that constitutes or characterizes a disease, condition, or disorder.

“Comprising” as used herein means “including, but not limited to”.

“Consisting” as used herein means “including and limited to”.

“Drug” or “therapeutic agent” as used herein can refer to any of a variety of drugs, pharmaceutical compounds, other bioactive agent that can be used as active agents to prevent or treat a disease.

“Bioabsorbable”, “biodegradable”, and “bioresorbable” are used herein synonymously to refer to a material or structure that degrades or dissolves in living tissues or systems of a body over time.

“Body lumen” as used herein refers to the cavity defined by a tubular structure of a mammalian body including, but not limited to, a blood vessel, a ureter, a urethra, a bile duct.

“Wall” as used herein refers to tissue that forms a tubular structure of a mammalian body including, but not limited to, a blood vessel wall, a ureter wall, a urethra wall, a bile duct wall.

“Scaffold” as used herein refers to a tubular structure that may be inserted into a body lumen. Scaffolds include stents that can insert into a blocked passageway to keep them open and restore the flow of blood or other fluids. Scaffolds also include devices that are not primarily intended to keep a blocked passageway open, but rather intended to divert flow of fluids. Scaffolds may also serve as a support for tissue growth such as neointimal growth. Scaffolds may also serve as a platform for the delivery of therapeutic agents. Scaffolds may be made of either metal or plastic.

“Visualization aid” as used herein refers to any structure that facilitates imaging by x-ray fluoroscopy.

“Resiliently deformable” as used herein pertains to an object that is capable of autonomously returning to its original shape upon release from a bent, stretched, compressed, or otherwise deformed shape.

“Endovascular device” as used herein refers to a prosthesis that can be implanted within a body lumen or body conduit.

“Fiber” as used herein refers to a filament, thread, tendril, or strand from which a textile is formed.

“Polymeric fiber” as used herein refers to fibers comprising a series of repeating monomeric units that have been cross-linked or polymerized. In some embodiments disclosed herein, only one polymer is used. In another embodiment, a combination of two or more polymers may be used. In another embodiment, polymers may be used with radio-opaque materials. The polymers and the combinations of polymers can be used in varying ratios to provide different properties. Polymers that may be used in the present invention include, for example, stable polymers, biostable polymers, durable polymers, inert polymers, organic polymers, organic-inorganic copolymers or inorganic polymers. Suitable polymers are bioabsorbable, biocompatible, bioresorbable, resorbable, degradable, and biodegradable polymers.

“Flow-diversion” as used herein refers to diversion of bodily fluid flow away from a pathology.

“Porosity” as used herein is, for a device in its fully expanded formation, the ratio of the free area to the total area, where the free area is equal to the total area minus the material surface area. In other words, the percentage of the overall device wall surface area that is open and fiber-free.

### DETAILED DESCRIPTION OF THE INVENTION

This disclosure generally relates to implantable devices, methods for manufacture and uses in either the prophylaxis or treatment of a pathology. Any term or expression not expressly defined herein shall have its commonly accepted definition understood by a person skilled in the art. To the extent that the following description is of a specific embodiment or a particular use of the invention, it is intended to be illustrative only, and not limiting of the invention, which should be given the broadest interpretation consistent with the description as a whole and with the claims.

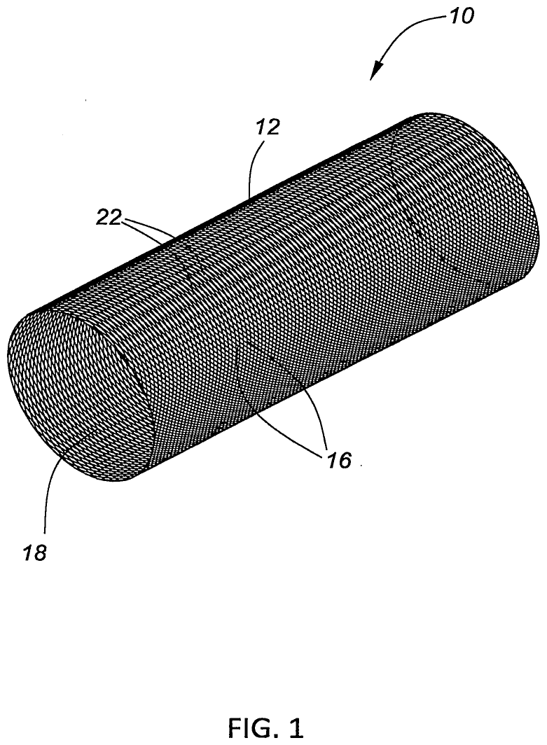

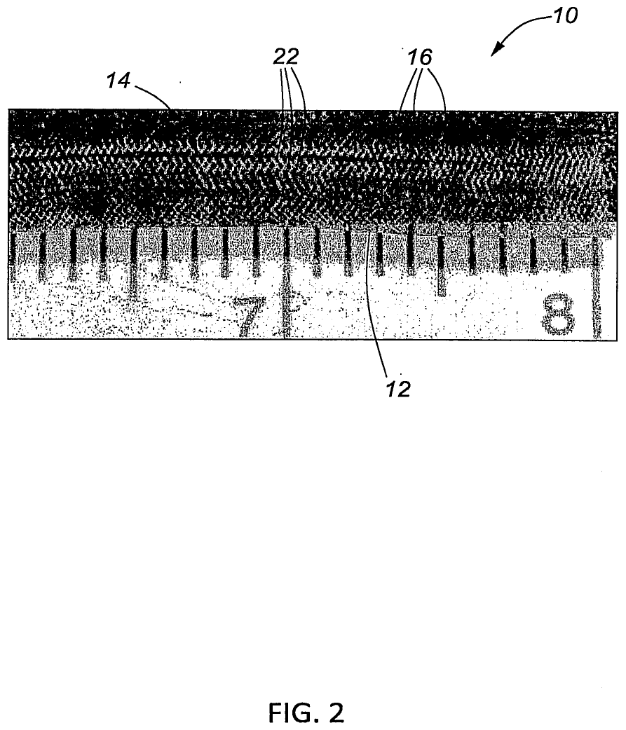

Referring to FIGS. 1 and 2, a device for positioning with a body lumen to achieve flow diversion of a bodily fluid according to a first embodiment of the invention is shown generally at 10. Referring to FIG. 2, device 10 comprises a resiliently deformable tubular body 12 formed of a braid 14 of interwoven bioabsorbable polymeric fibers 16. Referring to FIG. 1, tubular body 12 defines a lumen 18 through which a bodily fluid can continue to flow when device 10 is deployed within a body lumen. Overlapping bioabsorbable polymeric fibers 16 define pores 22.

In the presently described embodiment, braid 14 consists of 48 bioabsorbable polymeric fibers. However, flow diversion may be achieved with braids consisting of as few as 38 bioabsorbable polymeric fibers and as many as 96 bioabsorbable polymeric fibers. In various embodiments of the presently disclosed devices that are useful for flow diversion, a braid may comprise 40, 42, 44, 46, 48, 50, 52, 54, 56, 58, 60, 62, 64, 66, 68, 70, 72, 74, 76, 78, 80, 82, 84, 86, 88, 90, 92, or 94 bioabsorbable polymeric fibers. In particular embodiments of the presently disclosed devices that are useful for flow diversion, a braid may consist of 40, 42, 44, 46, 48, 50, 52, 54, 56, 58, 60, 62, 64, 66, 68, 70, 72, 74, 76, 78, 80, 82, 84, 86, 88, 90, 92, or 94 bioabsorbable polymeric fibers.

For applications where flow diversion is not necessary or desired, the braid of the presently disclosed invention could include as few as 20 bioabsorbable polymeric fibers, as few as 18 bioabsorbable polymeric fibers, as few as 16 bioabsorbable polymeric fibers, as few as 14 bioabsorbable polymeric fibers, or as few as 12 bioabsorbable polymeric fibers.

In the presently described embodiment, braid 14 consists of bioabsorbable polymeric fibers 16 having a diameter of 50 μm Bioabsorbable polymeric fibers useful for the production of devices useful for flow diversion as disclosed herein will have a diameter of at least about 30.mu.m, and will generally have a diameter in the range of about 30 μm to about 80 μm In various embodiments of the presently disclosed devices that are useful for flow diversion, the bioabsorbable polymeric fibers will have a diameter of about 30 μm, about 40 μm, about 50 μm, about 60 μm, about 70 μm, or about 80 μm. The skilled person will understand that bioabsorbable polymeric fibers with any diameter within this range may be useful in the production of a flow-diverting device.

For a flow diverting device, it is desirable for the tubular body to have a high flexibility so that it can be delivered through a microcatheter and, in various applications, through tortuous blood vessels and into the intracranial circulation. Accordingly, the upper limit of the diameter of the bioabsorbable polymeric fibers will be dictated by the desired flexibility of the tubular body as well as the diameter of the lumen into which the device is to be deployed.

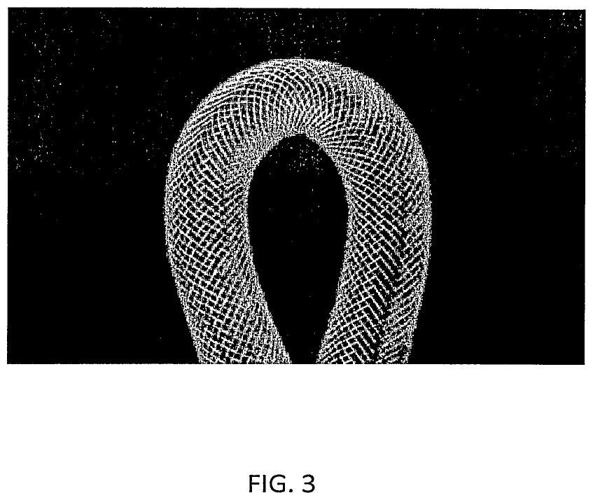

FIG. 3 is a picture demonstrating the flexibility and resilient deformability of the device consisting of 48 poly-L-lactic acid (PLLA) bioabsorbable fibers.

Porosity

The braided nature of the device is essential to flow diversion applications. The braid allows for the manufacture of a tubular body with a sufficiently high material surface area/sufficiently low porosity to prevent significant lateral flow of fluid through the side of the tubular body, thereby by allowing it to divert flow of fluid away from any site of interest that is spanned by the device. The braid also allows for collapsibility of the device within a microcatheter for delivery. Furthermore, the bioabsorbable polymeric fibers slide against each other, thereby facilitating expansion and retraction of the tubular body.

For flow diversion applications, porosity is the one of the most important design factors. Lower porosities result in a lower inlet and outlet velocity of blood flow into an aneurysm sac, thereby increasing the chance of thrombosis and faster occlusion. Decreasing the porosity of a BW stent also decreases wall shear stress (WSS) on both aneurysm and parent arterial wall. On the other hand, pressure in the dome of the aneurysm sac rises with decreasing porosity, thereby increasing the risk of aneurysm rupture associated with flow diverting scaffolds currently in clinical trials.

For flow diversion applications, a porosity of the tubular body in the range of about 60% to about 80% is desirable. In preferred embodiments, the porosity is in the range of about 60% to about 70% In various embodiments of the devices disclosed herein, the porosity will be about 60%, about 65%, about 70%, about 75%, or about 80%. In various embodiments, a pore density in the range of 10 pores/mm² to about 32 pores/mm² is desirable. In particular embodiments, the pore density is about 18 pores/mm². The skilled person will understand that as the porosity of the tubular body decreases, the flexibility/deformability of the tubular body may decrease. Accordingly, the limit to which porosity may be lowered is also informed by the required flexibility of the tubular body.

Pitch Angle

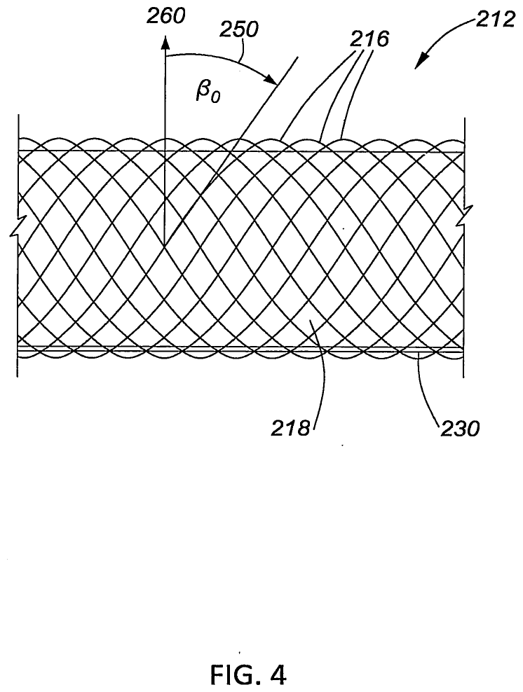

The pitch angle of the braiding process is an important factor influencing the material surface area and porosity of the tubular body in its expanded formation, and thus a device's flow diversion capability. The pitch angle further influences the resiliency of the device to deformation and thus self-expandability. Referring to FIG. 4, a resiliently deformable tubular body of a device according to an embodiment of the disclosure is shown generally at 212. Tubular body 212 comprises a plurality of bioabsorbable polymeric fibers 216. Overlapping bioabsorbable polymeric fibers 216 define pores 218. Tubular body 212 is depicted on a mandrel 230 as the braid is being manufactured. Pitch angle 250 of the braid is the angle formed between a bioabsorbable polymeric fiber 216 and the transverse axis 260 of tubular body 212.

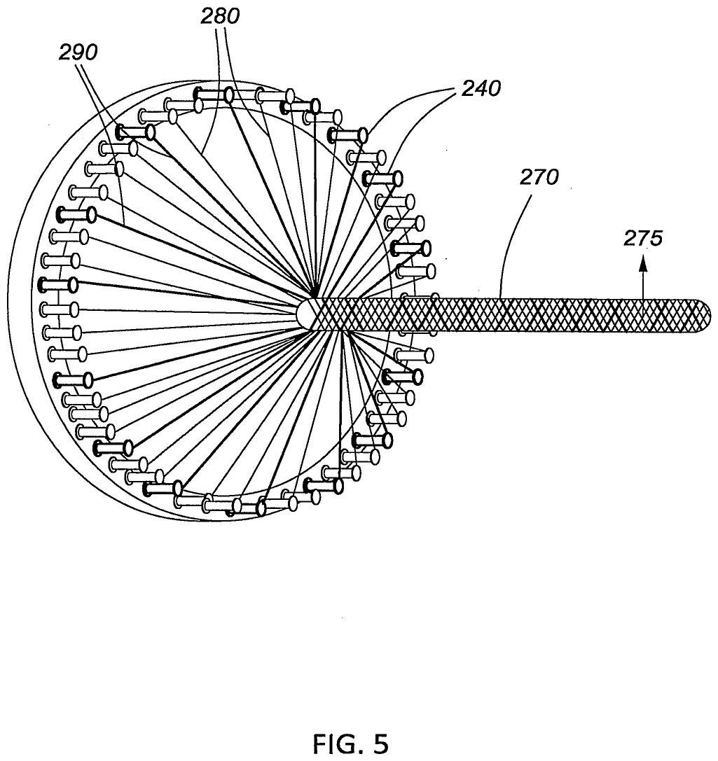

Referring, to FIG. 5, the pitch angle 260 of the braid is effectively determined by the angle formed between the bioabsorbable fibers 280 as they extend from carriers 240 to mandrel 250 and the transverse axis 270 of the mandrel 250.

The pitch angle, tubular body diameter factor, and bioabsorbable polymeric fiber diameter factor together to influence porosity of the tubular body and thus the ability of a device to divert flow. Accordingly, it is necessary to adjust these variables depending on the bioabsorbable polymeric fibers to be used or the tubular body diameter in order to achieve a porosity in the range of typical flow diverting device. For example, for a bioabsorbable polymeric fiber having a diameter of 50 μm and a desired tubular body diameter of 4 mm, the pitch angle should be about 16° or less, or about 15° or less. For a desired tubular body diameter of 5 mm, the pitch angle should be about 12° or less, or about 11° or less. For a desired tubular body diameter of 3 mm, the pitch angle should be about 18° or less, or about 17° or less. For a desired tubular body diameter of 7 mm, the pitch angle should be about 9° or less. Table 1 below provides general guidance on suitable combinations of tubular body diameter, fiber diameter, and pitch angle. However, the skilled person will understand that the combinations indicated are not intended to be limiting, and that it would be well within the purview of a skilled person to adjust each factor accordingly to achieve a suitable porosity.

TABLE 1
Suggested parameters for flow diverting devices of the disclosure.

| Tubular Body | Bioabsorbable Polymeric | Pitch angle |
|---|---|---|
| Diameter (mm) | Fiber Diameter (μm) | (gradian) |
| 3 | 40 | 16 |
| | 50 | 17-18 |
| 4 | 40 | 14 |
| | 50 | 15-16 |
| 5 | 40 | 10 |
| | 50 | 11-12 |

An achievable pitch angle is also dependent on the quality of the polymer fibers since it pitch angle imparts tension on the fibers that can potentially cause them to break. In general, a lower pitch angle allows for reduced porosity and a higher material surface area. By adding more fibers of a lower diameter, a lower pitch angle, and thus lower porosity, could be achieved for the device.

Bioabsorbable Polymeric Fibers

The polymer fibers used in the production of the disclosed devices comprise polymer material that is bioabsorbable. The polymeric material degrades in the body at a controlled/predictable rate and known period of time. The rate of degradation may depend on the polymer material, the diameter of the bioabsorbable polymeric fiber, physiological conditions, the porosity of the tubular body, etc.

Referring back to FIG. 2, the bioabsorbable polymeric fibers 16 of the depicted embodiment comprise poly-L-lactic acid (PLLA). However, any one or more of a plurality of bioabsorbable polymeric fibers could be utilized including fibers comprising polylactides (PLA), polyglycolides (PGA), polycaprolactone (PCL), polylactide-co-glycolides (PLGA), polyanhydrides, polyorthoesters, poly(N-(2-hydroxypropyl) methacrylamide), poly(l-aspartamide), DLPLA-poly(dl-lactide), poly (L-Lactic acid); LPLA-poly(l-lactide), PDO-poly (dioxanone), PGA-TMC-poly (polyglycolide-co-trimethylene carbonate), PGA-LPLA-poly(l-lactide-co-glycolide), PGA-DLPLA-poly(dl-lactide-co-glycolide), LPLA-DLPLA-poly(l-lactide-co-dl-lactide), PDO-PGA-TMC-poly(glycolide-co-trimethylene carbonate-co-dioxanone), or any combination thereof.

In some applications, it may be desirable to induce an inflammatory response in the vicinity of the tissue proximal to the deployed device so as to promote the formation of scar tissue. For example, in flow diversion applications directed at treating an aneurysm, the promotion of scar tissue in the blood vessel wall at the neck of the aneurysm as it heals may improve the strength of the vessel at the site and reduce the risk that an aneurysm will redevelop. For such applications, embodiments employing a bioabsorbable polymeric fiber that forms lactic acid upon degradation may be useful. Accumulating acidic degradation products decrease the pH of the surrounding tissue, which may trigger inflammatory and foreign body reactions at the site of the pathology. Implantation of PLLA scaffolds in the coronary arteries of mini-pigs results in expression of NF-kB a marker of inflammation that mediates expression of numerous inflammatory cytokines. Accordingly, particular embodiments of the invention may utilize bioabsorbable polymeric fibers that comprise polylactides (PLA), polylactide-co-glycolides (PLGA), DLPLA-poly(dl-lactide), poly (L-Lactic acid); LPLA-poly(l-lactide), PGA-LPLA-poly(l-lactide-co-glycolide), PGA-DLPLA-poly(dl-lactide-co-glycolide), LPLA-DLPLA-poly(l-lactide-co-dl-lactide), or any combination thereof.

The devices disclosed herein display special structural features when axially extended/expanded or compressed. When expanded, the structure is capable of substantially accommodating strain or stress forces since the initially inclined fibers are free to pivot to a position parallel to the direction of the stress. In addition, individual polymeric fibers may slide up against each other providing elastic and flexible properties to the device.

Visualization Aids

This braided assembly exhibits special structural features when axially extended or compressed. When extended, the structure is capable of substantially accommodating strain or stress forces since the initially inclined fibers are free to pivot to a position parallel to the direction of the stress. In addition, individual polymeric fibers may slide up against each other providing elastic and flexible properties to the device.

It is critical for the physician deploying a device within a body lumen to be able to determine the position of the device within the lumen. Thus, it is desirable for devices as disclosed herein to include a visualization aid. Accordingly, various embodiments of the implantable devices disclosed herein will comprise a radio-opaque material to facilitate imaging of the device in the body lumen by X-ray fluoroscopy.

Such radio-opaque materials may include tantalum, platinum, tungsten, gold, iodine, or combinations thereof. The radio-opaque material may be selected according to the polymeric material of the bioabsorbable polymeric fiber, the imaging technology, the pathology to be treated, etc.

A radio-opaque material may be attached or in contact with polymeric fibers in various ways, for example by covalent bonding of a radio-opaque material with a bioabsorbable polymeric fiber, adhesion of a radio-opaque material to a bioabsorbable polymeric fiber, or other forms of attachment, contact, bonding, blending or incorporation of the radio-opaque material with the polymeric fibers.

In presently preferred embodiments, a plurality of individual radio-opaque metal wires, typically six or eight, are first braided with a multiplicity of bioabsorbable polymer filaments, typically 40 or 42, After cutting the ends of the metal wires and the bioabsorbable polymer filaments from the braiding machine to form the stent structure, the free ends of the radio-opaque metal wires are joined together at both ends of the stent. The free ends may be joined by any one or more of welding, fusing, swaging, or soldering. In preferred embodiments, the free ends of the radiopaque metal wires may be welded in radiopaque cylindrical marker bands to further improve fluoroscopic imaging during delivery. The radio-opaque metal wires and the bioabsorbable fibers preferably remain free at their cross-over points. The free ends of the polymer wires are typically not attached.

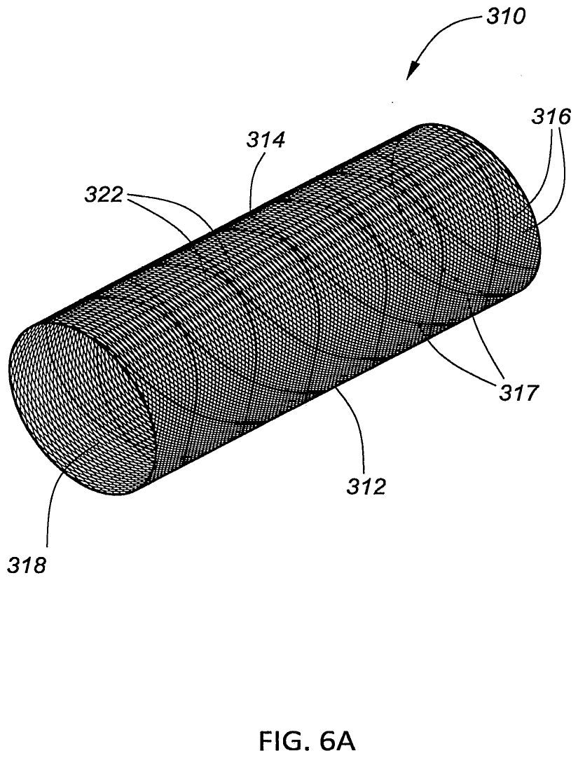

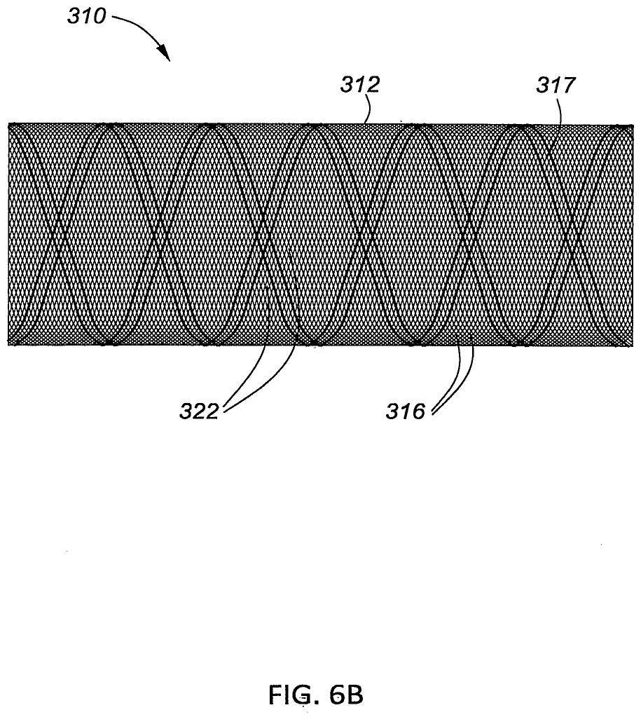

Referring to FIGS. 6A, 6B, 7A, 7B, 8A, and 8B, a device for positioning with a body lumen to achieve flow diversion of a bodily fluid according to a second embodiment of the invention comprising a visualization aid is shown generally at 310. Device 310 comprises a resiliently deformable tubular body 312 formed of a braid 314 of interwoven bioabsorbable polymeric fibers 316. Referring to FIG. 1, tubular body 312 defines a lumen 318 through which a bodily fluid can continue to flow when device 310 is deployed within a body lumen. A visualization aid is provided by four radio-opaque wires 317 that are interwoven with bioabsorbable polymeric fibers 316 to form part of braid 314. Overlapping bioabsorbable polymeric fibers 316 and radio-opaque wires 317 define pores 322.

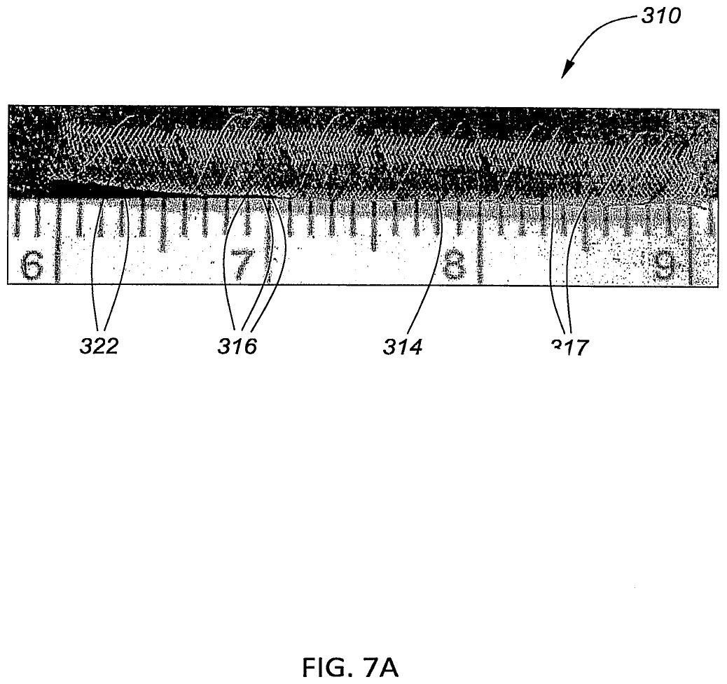

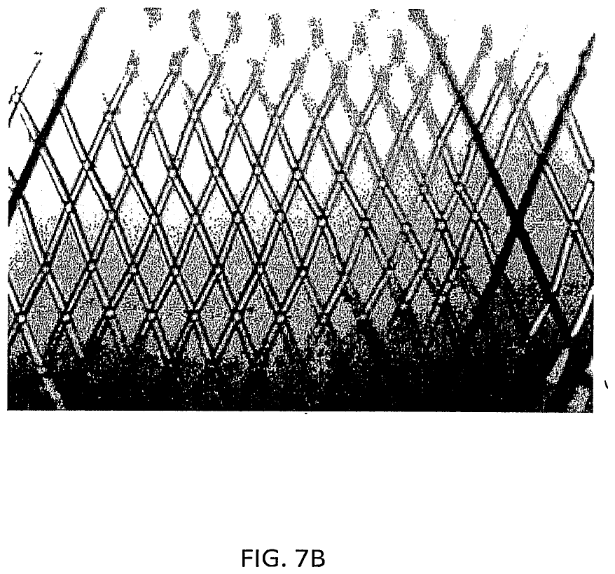

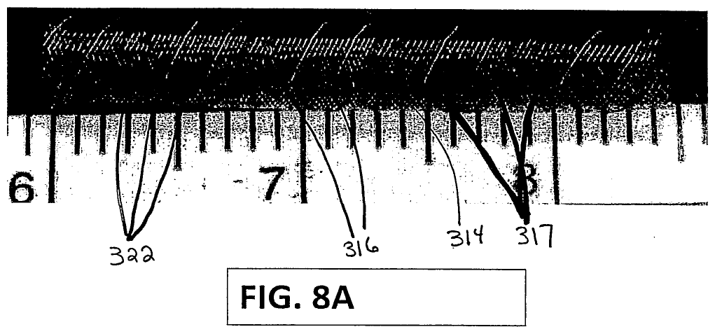

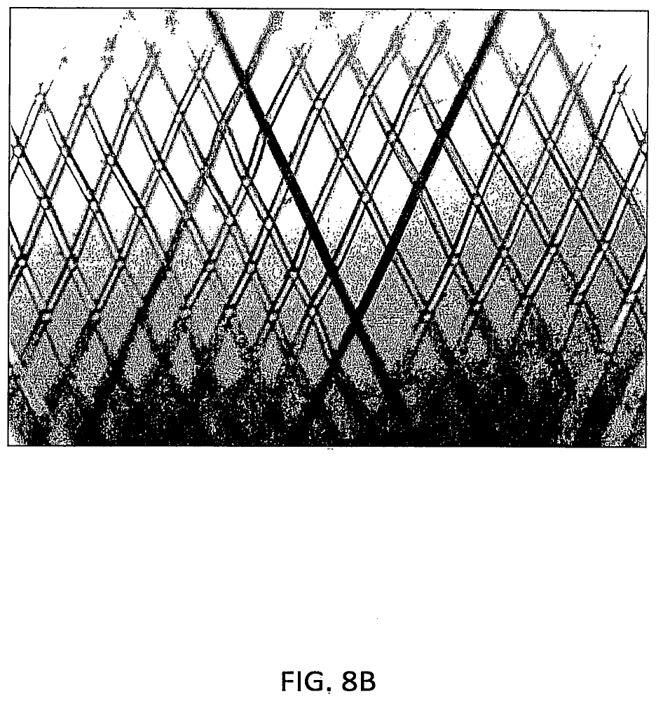

The embodiment depicted in FIG. 7 utilizes 44 bioabsorbable polymeric fibers and 4 radio-opaque wires. The embodiment depicted in FIG. 8A utilizes 46 bioabsorbable polymeric fibers and 2 radio-opaque wires. However, any number of radio-opaque wires could be used as a visualization aid. The number used may depend on a variety of factors including the nature of the radio-opaque material. As few as a single radio-opaque wire may be sufficient. However, the ability to visualize the device improves with the number of radio-opaque wire utilized. In various embodiments, 2, 3, 4, 5, 6, 7, 8, 9, 10, or 12 radio-opaque wires may be utilized. Preferably, an even number of radio-opaque wires is utilized to maintain balance. In preferred embodiments, 6 radio-opaque wires or 8 radio-opaque wires are utilized. The skilled person will understand that resolution of the device may decrease with increasing number of radio-opaque wires and thus the selected number will reflect a balance between detectability and sharpness of the image.

As indicated above, the radio-opaque wires 317 may comprise radio-opaque materials such as tantalum, platinum, tungsten, gold, iodine, or combinations thereof. In particular embodiments, the radio-opaque wires may be resiliently deformable. In some embodiments, the resiliently deformable wires are made from a nickel-titanium alloy (e.g. nitinol), a cobalt-chromium alloy (e.g. Phynox), or a cobalt-chromium-nickel alloy. Each resiliently deformable wire may independently be made of a nickel-titanium alloy coated with the radio-opaque material, a drawn filled tube (DFT) comprising a nickel-titanium alloy exterior and a core comprising the radio-opaque material, a DFT comprising an exterior comprising the radio-opaque material and a core comprising a nickel-titanium alloy, a cobalt-chromium-nickel alloy coated with the radio-opaque material, a DFT comprising a cobalt-chromium-nickel alloy exterior and a core comprising the radio-opaque material, or a DFT comprising an exterior comprising the radio-opaque material and a core comprising cobalt-chromium-nickel alloy. In particular embodiments, the radio-opaque wire is a tantalum-coated nitinol wire. In other embodiments, the radio-opaque wire comprises a DFT having a nitinol exterior and a platinum core.

Facilitating and Maintaining Expansion

It is important that, upon deployment in a lumen, the exterior surface of the tubular bodies of the presently disclosed devices remains closely appressed to the body wall, particularly in devices for flow diversion applications in blood vessels. If the exterior surface of the tubular body is not closely appressed to the blood vessel wall, thromboses will form in the spaces between the tubular body and the blood vessel wall, and lead to occlusion of the blood vessel. While embodiments of the devices disclosed herein that include only bioabsorbable polymeric fibers are resiliently deformable, they may be at prone to shrinkage or partial collapse within the blood vessel. Moreover, the bioabsorbable polymeric fibers may have a tendency to lose some of their ability to self-expand when stored in a compressed state for a prolonged period of time.

Accordingly, various embodiments of the devices disclosed herein include means for facilitating and/or maintaining radial expansion of the tubular body in the body lumen so as to maintain the exterior surface of the tubular body closely appressed to the body wall. Such means also assist in facilitating and/or maintaining axial expansion of the device. Accordingly facilitating and/or maintaining radial and/or axial expansion may contribute to self-expansion of the device upon deployment in the lumen.

The means for facilitating and/or maintaining radial and/or axial expansion of the tubular body in the body lumen may include a wire interwoven with the plurality of bioabsorbable polymeric fibers to form part of the braid. In operation, the wire exerts a radial force on the tubular structure to facilitate radial expansion upon deployment and to urge the tubular structure against the body wall to maintain the tubular structure in fully expanded form and appressed to the body wall. In particular embodiments, the wire is resiliently deformable. The resiliently deformable wire may comprise a nickel-titanium alloy or a cobalt-chromium-nickel alloy.

As few as a single wire may be sufficient to facilitate and maintain radial and/or axial expansion of the tubular body. However, the radial force exerted by the tubular body as it expands will increase with the number of wires used. In various embodiments, 2, 3, 4, 5, 6, 7, 8, 9, 10, or 12 radio-opaque wires may be utilized. Preferably, an even number of radio-opaque wires is utilized to maintain balance. In preferred embodiments, 6 radio-opaque wires or 8 radio-opaque wires are utilized.

It will be readily apparent to the skilled person that the same wires may be used as both a visual aid and as a means for facilitating and/or maintaining radial and/or axial expansion. Accordingly, the wires may comprise radio-opaque materials such as tantalum, platinum, tungsten, gold, iodine, or combinations thereof. In particular embodiments, the radio-opaque wires may be resiliently deformable. In some embodiments, the resiliently deformable wires are made from a nickel-titanium alloy (e.g., nitinol), a cobalt-chromium alloy (e.g., Phynox®), or a cobalt-chromium-nickel alloy. Each resiliently deformable wire may independently be made of a nickel-titanium alloy coated with the radio-opaque material, a drawn filled tube (DFT) comprising a nickel-titanium alloy exterior and a core comprising the radio-opaque material, a DFT comprising an exterior comprising the radio-opaque material and a core comprising a nickel-titanium alloy, a cobalt-chromium-nickel alloy coated with the radio-opaque material, a DFT comprising a cobalt-chromium-nickel alloy exterior and a core comprising the radio-opaque material, or a DFT comprising an exterior comprising the radio-opaque material and a core comprising cobalt-chromium-nickel alloy. In particular embodiments, the radio-opaque wire is a tantalum-coated nitinol wire. In other embodiments, the radio-opaque wire comprises a DFT having a nitinol exterior and a platinum core.

Accordingly, a metal wire component may provide at least three independent advantage, namely: 1) allowing for radio-opacity and thus visualization by means of X-ray fluoroscopy; 2) improving self-expandability, and 3) improving radial force to maintain radial expansion (crush force and chronic outward force) to maintain the outer wall of the tubular body closely appressed to the body wall.

Manufacture

Referring back to FIGS. 4 and 5, a device as disclosed herein may be formed, for example, from individual interwoven bioabsorbable polymeric fibers and, in various embodiments, radio-opaque wires to create a braid forming the tubular body.

Braiding the tubular bodies on, for example, a “maypole style” machine avoids the need for known laser cutting techniques for manufacturing a device for deployment in a body lumen. Instead, bioabsorbable polymeric fibers of varying diameters may be braided on a mandrel at varying pitch angles to produce braided, hollow, and tubular bodies with varying porosities. The braid may be a linear fibrous assembly with sets of interlacing bioabsorbable fibers that lie on a bias relative to the longitudinal axis of the structure. The braiding may be clockwise or counter-clockwise interlacing or spiraling fibers.

Several patterns of braids or interlacing fibers may be used. The present invention is not limited to any of the following examples: a “1-over-1-under-1” or “half load” pattern; a “2-under-2-over-2” or “diamond” pattern; a “1-under-2-over-2” (otherwise known as “1-over-2-under-2”) or “full load” pattern; or other variations.

For the 1-under-2-over-2 pattern, a 48 carrier machine can be used to produce a 48 fiber design. For a 1-over-1-under-1 pattern, a 96 carrier machine is required for the design that also still comprises 48 fibers. The desired pattern may depend on several factors including tubular body width, bioabsorbable polymeric fiber diameter, and the particular bioabsorbable polymeric. For example, 2-under-2-over-2 increases braid thickness and thus influences the choice of possible tubular bodies that can be made with this pattern.

Referring to FIG. 5, and as described above, the pitch angle of the braid is the angle formed by between bioabsorbable polymeric fibers 280 (or wires 290), as they extend from carriers 250 to mandrel 270, and the transverse axis 275 of the mandrel 270 (i.e. the perpendicular axis to the longitudinal direction of mandrel 270).

Referring still to FIG. 5, in embodiments that involve optionally radio-opaque wires, wires 290 are preferably loaded as pairs on opposing carriers 240 to that forces are balanced within the braided product.

With respect to embodiments disclosed herein that involve resiliently deformable radio-opaque wires that require heat treatment to set the original shape of the wire, it may not be necessary or desired to set the shape of the wire in some embodiments. However, where it is desired to set the original shape of the wire, it is important to note that they should not be shape set (or “annealed”) straight, as this would adversely affect the lower radial exerted by the tubular body upon expansion, and result in an inability to cause the tubular body to adequately expand after being deformed or delivered through a catheter. Thus, it is preferable to shape set the wire on the mandrel. However, it is undesirable to shape set the wire on the mandrel with the bioabsorbable polymeric fibers because, in order to shape set the wire, it is necessary to heat the wire to a temperature of upwards of 500 degrees Celsius, which would melt the bioabsorbable polymer fibers if they were on the mandrel at the same time as the wires. One option may be to shape set the wired on a mandrel without the polymer fibers. The shape set wire could then be rewound into the bobbin and then braided with the bioabsorbable polymeric fibers. Another option may be to shape set the final braided design at a lower temperature (e.g.) in order to relieve any residual stress on the polymer fibers. This would essentially shape set the bioabsorbable polymer fibers, but not the radio-opaque wires, in the final design. As described above, another option is to simply forgo shape setting the wire or bioabsorbable polymeric fibers.

Some metal wires may flare out at the ends of the scaffold upon production, which could result in puncture of the body wall (e.g. a blood vessel) upon delivery. The flaring of the metal wires could also depend on where the scaffold is cut from the mandrel. For example, if the scaffold is cut precisely at the point where two metal wires overlap, there will likely be less flare-out. Accordingly, it may be preferable in some embodiments to solder the metal wires together. Fusing, welding, swaging, soldering or otherwise connecting the ends of the metal wires, as described above, will usually reduce such flaring and can also inhibit or prevent the “curling” of the stent which can occur if the metal wire ends are left unconnected.

Therapeutic Agent Delivery

The devices disclosure herein may also be useful for delivering a therapeutic agent to a pathology of or proximal to a body wall defining the lumen. The bioabsorbable polymeric fibers of the tubular body may be coated with or conjugated to the therapeutic agent, or the therapeutic agent may be incorporated within the bioabsorbable polymeric fiber. The therapeutic agent may be slowly released over time to treat the pathology. In the context of an endovascular device for implantation in a blood vessel, the therapeutic agent may be an antibiotic agent, an antiviral agent, an analgesic, a muscle relaxant, a chemotherapeutic agent, an intra-arterial vasodilating agent, a calcium channel inhibitor, a calcium channel antagonist, a calcium channel blocker, a transient receptor potential protein blocker, an endothelin antagonist, a blood thinning agent, an antiplatelet agent, or any combination thereof.

In various embodiments, the therapeutic agent may include paclitaxel, sirolimus, everolimus, temozolamide, cyclophosphamide, doxorubicin, irinotecan, azathioprine, methotrexate, cisplatin, or vincristine. In the particular context of a flow diverting device as disclosed herein for treatment of an aneurysm, the therapeutic agent may include one or more blood thinners/antiplatelet agents such as aspirin, heparin, Ticagrelor, 5-fluorouracil, melphalan, or clopidogrel.

The therapeutic agents may also be used in the form of their pharmaceutically acceptable salts or derivatives and in the case of chiral active ingredients. It is also possible to employ both optically active isomers and racemates or mixtures of diastereoisomers. As well, a therapeutic agent may include a prodrug, a hydrate, an ester, a derivative or analogs of a compound or molecule.

As discussed above, the polymeric material itself may, in some contexts, provide lactic acid upon degradation, which may aid in healing and strengthening body wall at the site of the pathology such as an aneurysm.

The therapeutic agents may elute over a controlled period of time, which is shown to be effective, to minimize side effects. A device as disclosed herein may be placed at a site proximal to the pathology. In this way, the therapeutic agent can be targeted to the disease while side effects may be minimized, as the therapeutic agent may not be distributed to organs that do not involve the disease, as in the case of oral administration or intravenous administration of therapeutic agent.

At least two mechanisms may regulate the release kinetics of a therapeutic agent: 1) a diffusion-controlled mechanism, in which the therapeutic agent diffuses outwardly through the bulk polymer due to a concentration gradient, and 2) a degradation-controlled mechanism, in which release of the therapeutic agent depends on the hydrolytic or other degradation of the polymeric material and erosion of polymeric fiber surface.

A device of the present disclosure may be configured so that the initial release of the therapeutic agent can be deferred to correspond to the delayed clinical manifestations of the disease. The desired timing of therapeutic agent release may vary, for example, it may be immediate for patients who already have a disease. A device may alternatively be used prophylactically in patients who are at high risk of developing a disease or pathology, in which case the desired timing of drug release may be delayed.

A device of the present disclosure may also be configured so that the release of the therapeutic agent is triggered by the introduction of another therapeutic agent, a physiological condition, or any change within the bodily lumen.

Operation

The presently disclosed devices comprising resiliently deformable tubular bodies may self-expand when deployed within a bodily lumen. The degree of expansion may depend on the polymeric material, crystallinity of the polymer, diameter of the polymeric fiber, diameter of the tubular body, pitch angle of the weave, physiological conditions, polymer annealing temperature or the structural contribution of any included material such as a radio-opaque material or similar parts. Various embodiments of the devices disclosed herein may exhibit memory self-expansion in the body.

The resiliently deformable and self-expanding features of the tubular bodies of the devices disclosed herein allow them to be configured in a radially compressed state for intraluminal catheter implantation. Once properly positioned adjacent the pathology in the body lumen, the device is allowed to expand radially and axially such that the outer surface of the tubular body becomes appressed to the body wall defining the lumen. Radial expansion of the device may be assisted by inflation of a balloon attached to the catheter.

The devices disclosed herein may be pre-loaded in a kit, for example in a sheath or a micro-catheter for ease of delivery or for immediate deployment. The kit may include a device as disclosed herein pre-loaded within a delivery system suitable for inserting the device into a patient, delivering the device through the lumen of a body, e.g. the vascular system of a patient, and deploying the device to the desired position for implantation of the device within the body of the patient. The delivery system may include a sheath, a catheter, a guide wire, and/or any other elements for insertion, delivery, guiding, deployment, and implantation of the vascular device, or combinations thereof.

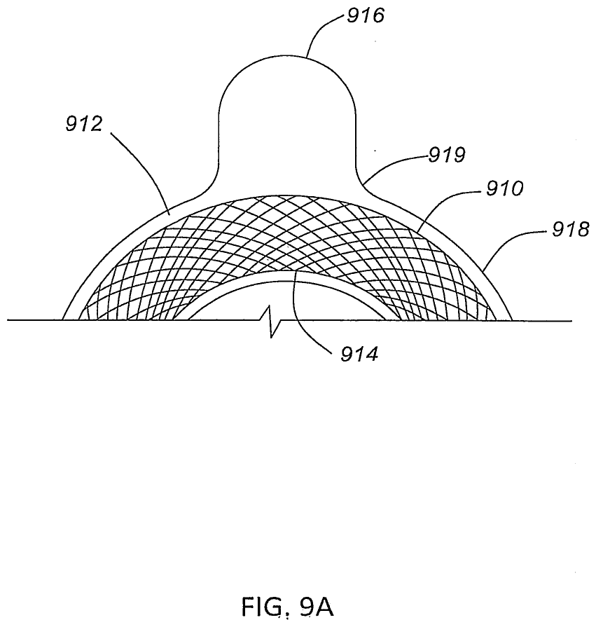

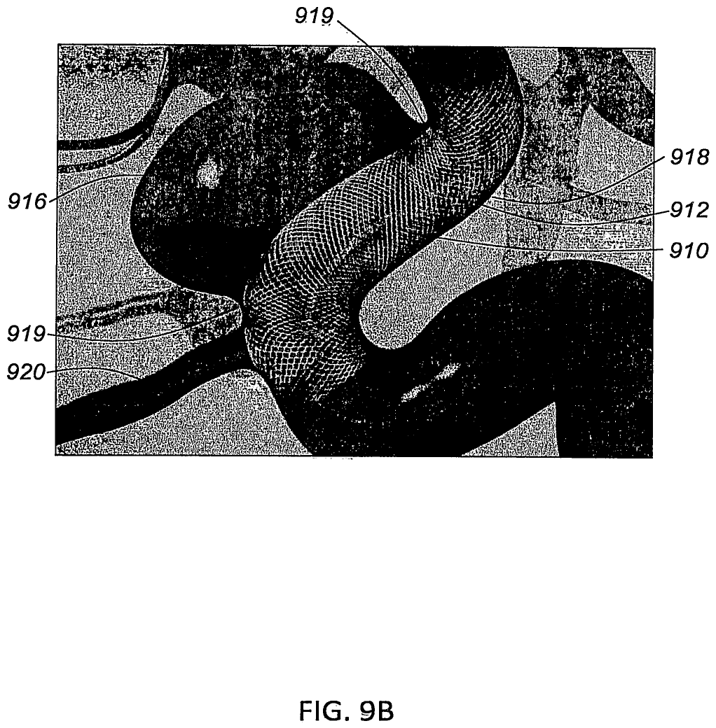

According to one embodiment of the disclosure, an endovascular device of may be configured to divert blood flow away from the downstream intravascular territory or the site of a disease. In particular, diversion of blood through the vascular network may be necessary to prevent or treat an unruptured or ruptured brain aneurysm. Referring to FIGS. 9A and 9B, endovascular device 910 is thus deployed in the lumen 912 defined by blood vessel wall 918 proximal to the aneurysm 916 and allowed to expand such that, when tubular body 914 is full expanded, the outer surface of the tubular body is closely appressed to the blood vessel wall 918 and spans the neck 919 of the aneurysm. The low porosity of the braid thus diverts flow of blood past the neck of the aneurysm 916. At the same time, the braid is sufficiently porous to permit a small amount of blood to enter the aneurysm sac with low velocity, which causes thrombosis and occlusion of the aneurysm, and permits the aneurysm to heal. Referring to FIG. 9B, the braid is also sufficiently porous to permit enough blood to flow throw through the pores to healthy blood vessel branches, e.g. branch 920, that may also be spanned, or partially spanned, by the device, thereby maintaining their patency.

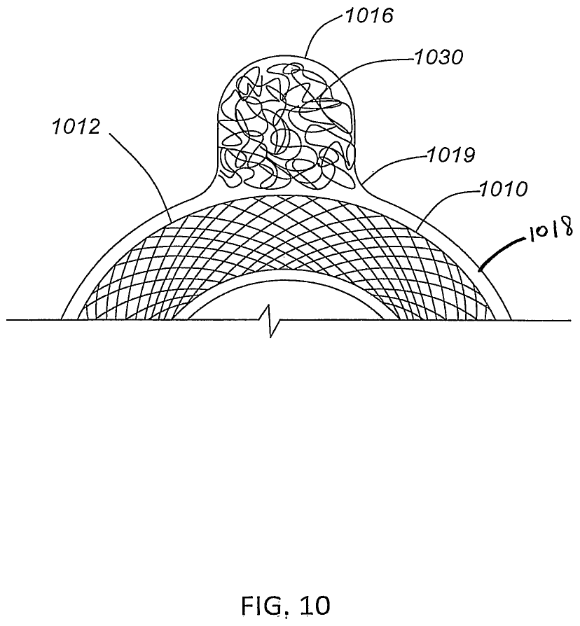

In another embodiment, an endovascular device according to an embodiment disclosed herein may be used to support coils placed into the aneurysm to prevent prolapse into a parent blood vessel, for example by aneurysm-bridging. The endovascular device may be configured to fit into a bodily lumen in combination with metal coils or a balloon. Referring to FIG. 10, the aneurysm neck 1019 may be wide. In such circumstances the endovascular device 1010 can serve to remodel the neck 1019 and support the metal coils 1030 placed into the aneurysm 1016. The endovascular device can prevent the metal coils from traveling within the body lumen 1012 defined by blood vessel wall 1018, for example preventing the coils from entering a parent blood vessel. After the procedure, the endovascular device 1010 will typically be left in place, but may be removed in some embodiments. In another embodiment, the endovascular device may be configured to fit into a bodily lumen to support the metal coils in any manner.

### EXAMPLES

While specific embodiments of the invention have been described and illustrated, such embodiments should be considered illustrative of the invention only and not as limiting the invention as construed in accordance with the accompanying claims.

### Example 1

Referring to FIG. 4, a device was made constructed with 48 bioabsorbable polymeric fibers of poly-L-lactic acid with a molecular weight of 30,000 g/mol and a diameter of 50 μm Example 2

Referring to FIG. 7, a device was made constructed with 44 bioabsorbable polymeric fibers of poly-L-lactic acid with a molecular weight of 30,000 g/mol and a diameter of 50 μm interwoven with four radio-opaque fibers of tantalum-coated nitinol. The device was tested in animal blood vessels, i.e. rabbit aortas, and was able to keep important vascular side branches open without occluding any of the blood vessels.

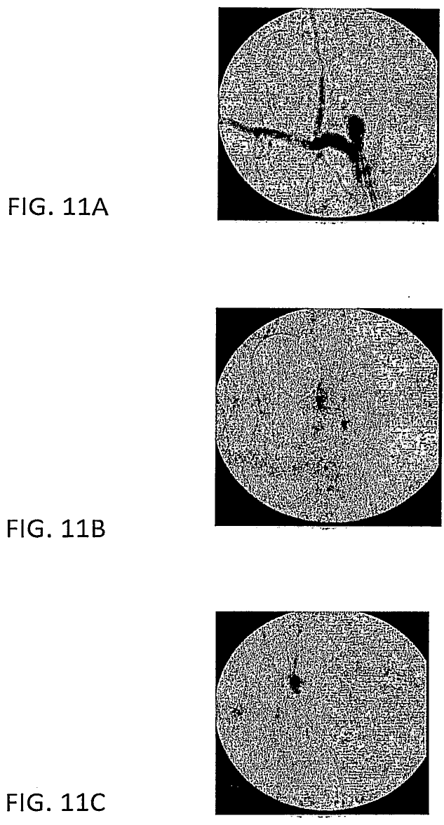

FIGS. 11A and 11B are time lapse photos of an angiogram of an aneurysm during early arterial and early venous phase prior to implantation of the device. The rapid washout of signal from the aneurysm shown in FIG. 11B is indicative of fluid flow into the aneurysm. In contrast, FIG. 11C shows early venous phase after implantation of the device, wherein signal is retained in the aneurysm. This indicates that blood is no longer flowing freely into the aneurysm and that the device is successfully diverting flow from the aneurysm.

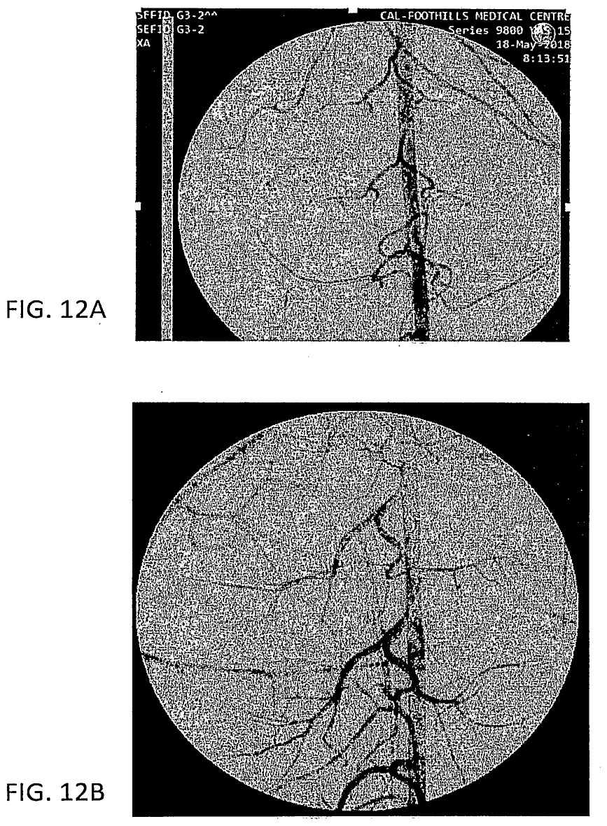

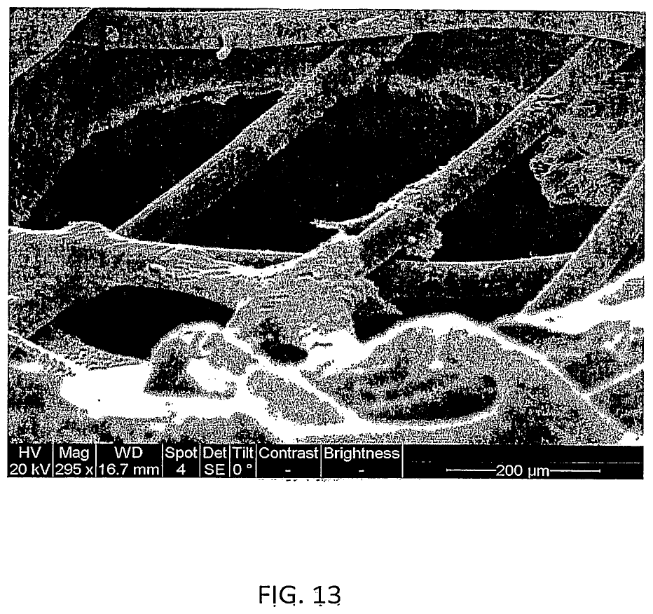

Referring to FIGS. 12A and 12B, rabbit aortas into which the device was deployed showed persistent angiographic patency of the aorta where the device was placed as well as the “jailed” side branches after 1 month (FIG. 12B). FIG. 13 is a scanning electron micrograph of showing persistent patency of a side branch of the rabbit aorta after 1 month implantation of the device.

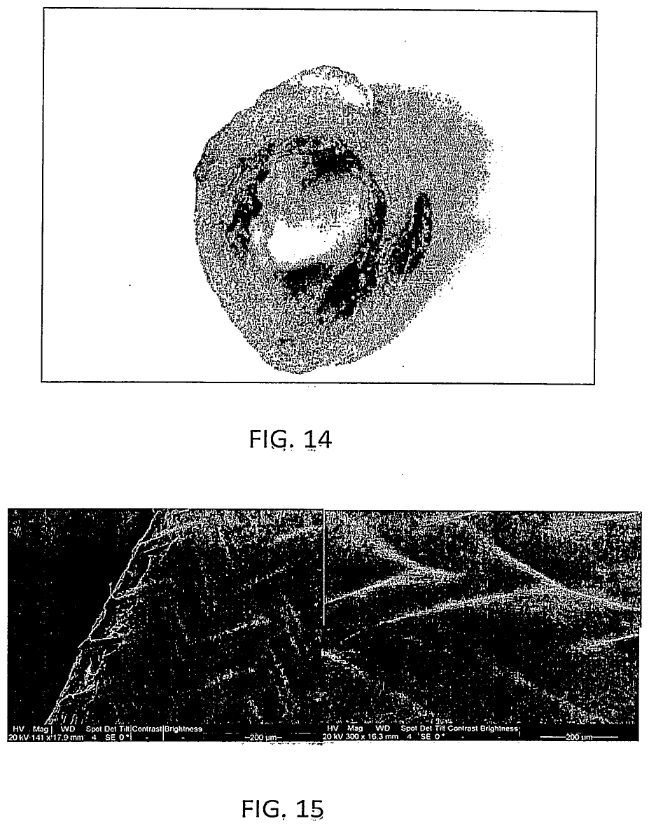

Referring to FIG. 14, the device showed excellent blood vessel wall apposition.

FIG. 15 is scanning electron micrographs of showing a smooth neointimal layer forming over interior surface of the tubular body 1 month after implantation of the device.

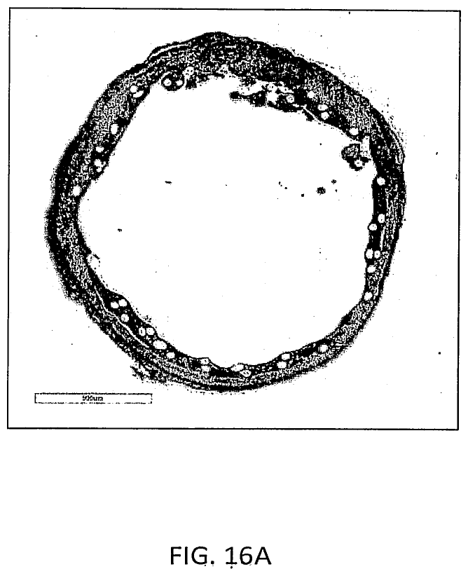

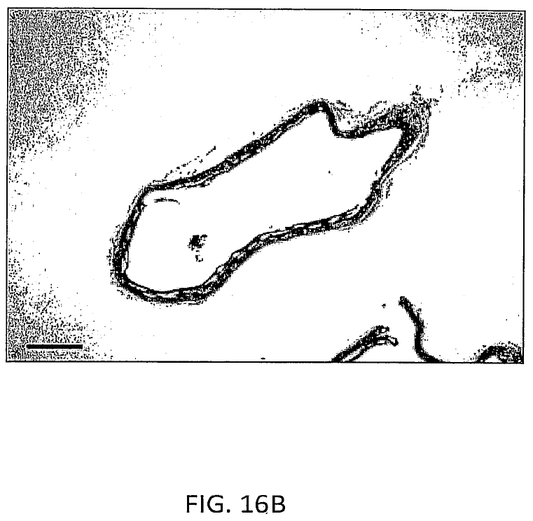

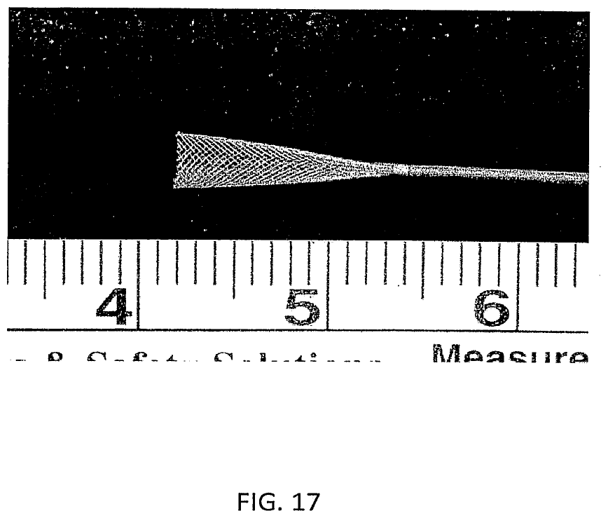

FIG. 16A is a histological cross section of a rabbit aorta showing persistence of polymer fibers and neointima formation over the fibers one month after implantation of a device.

FIG. 16B is a histological cross section of a rabbit aorta showing persistence of polymer fibers, neointima formation over the fibers, and a lack of exuberant inflammatory response two months after implantation of a device.

The lack of an exuberant inflammatory response on histology at 2 months is believed to be due to the thin diameter of the bioabsorbable polymeric fibers (roughly 50 microns). The presently disclosed scaffolds contrast with the thick struts of the previously FDA approved laser-cut bioabsorbable stent (marketed and sold by Abbott Vascular as the Absorb BVS stent).

The formation of the neointima over the interior surface of the interior body, the lack of an exuberant inflammatory response as indicated by histology at 2 months, demonstrates the biocompatibility of the device with the blood vessel wall. Response of the blood to the polymer material is important because it can result in unwanted thrombosis or hemolysis. The thrombogenicity of the device was compared to that of the leading metal flow diverting device (i.e. Pipeline™) in terms of thrombotic response. The device of the present disclosure showed a lower % thrombosis surface coverage as well as a lower hemolytic index compared to, i.e,. Pipeline™, as indicated in Table 2 and Table 3.

Table 2 shows a lower % thrombosis surface coverage for the device of the present disclosure compared with Pipeline™ (tests done as per ISO standards).

TABLE 2

| Sample type | % lumen | % thrombosis |
|---|---|---|
| (N = 3 for each) | occlusion | surface coverage |
| Positive control | 100% | 100% |
| Negative control | 0% | 0% |
| Comparative sample | 0% | 3.6% |
| (Pipeline ™) | | |
| Bioabsorbable Stent | 0% | 2.3% |

Table 3 provides the results of in vitro hemolysis studies (performed according to ASTM standards), showing a lower hemolytic index of the presently disclosed device compared with Pipeline™.

TABLE 3

| | | Plasma | Total | | |
|---|---|---|---|---|---|
| | | Hemo- | hemo- | | Mean |
| | Repli- | globin | globin | Hemolytic | Hemolytic |
| Experiment Type | cate | (mg/ml) | (mg/ml) | Index | Index |
| Pipeline (Predicate) | 1 | 1.11 | 185.74 | 0.5 | 0.5 |
| Control | 2 | 0.88 | 211.56 | 0.8 | |
| | 3 | 1.21 | 196.35 | 0.6 | |
| Negative Control | 1 | 1.11 | 185.74 | 0.03 | 0.02 |
| (glass) | 2 | 0.88 | 211.56 | 0.01 | |
| | 3 | 1.21 | 196.35 | 0.02 | |
| Positive Control | 1 | 1.11 | 185.74 | 12.9 | 15.5 |
| | 2 | 0.88 | 211.56 | 15.6 | |
| | 3 | 1.21 | 196.35 | 17.9 | |
| Bioabsorbable | 1 | 1.11 | 185.74 | 0.4 | 0.4 |
| Stent | 2 | 0.88 | 211.56 | 0.6 | |
| | 3 | 1.21 | 196.35 | 0.2 | |

Without wishing to be bound by theory, it is believed that the small diameter of the bioabsorbable polymeric fibers (about 50 μm) contributes to this observed biocompatibility. In comparison, the comparatively thick polymeric fibers of previously FDA approved, laser-cut bioabsorbable devices having fibers of about 150 μm in diameter (marketed and sold by Abbott Vascular as the Absorb BVS) were prone to causing thrombosis (see Expert Opin Drug Deliv. 2016 October; 13(10):1489-99).

### Example 3

Referring to FIG. 8A a device was made constructed with 46 bioabsorbable polymeric fibers of poly-L-lactic acid with a molecular weight of 30,000 g/mol and a diameter of 50 μm interwoven with two radio-opaque fibers of tantalum-coated nitinol. The device was tested in animal blood vessels and was able to keep important vascular side branches open without occluding any of the blood vessels.

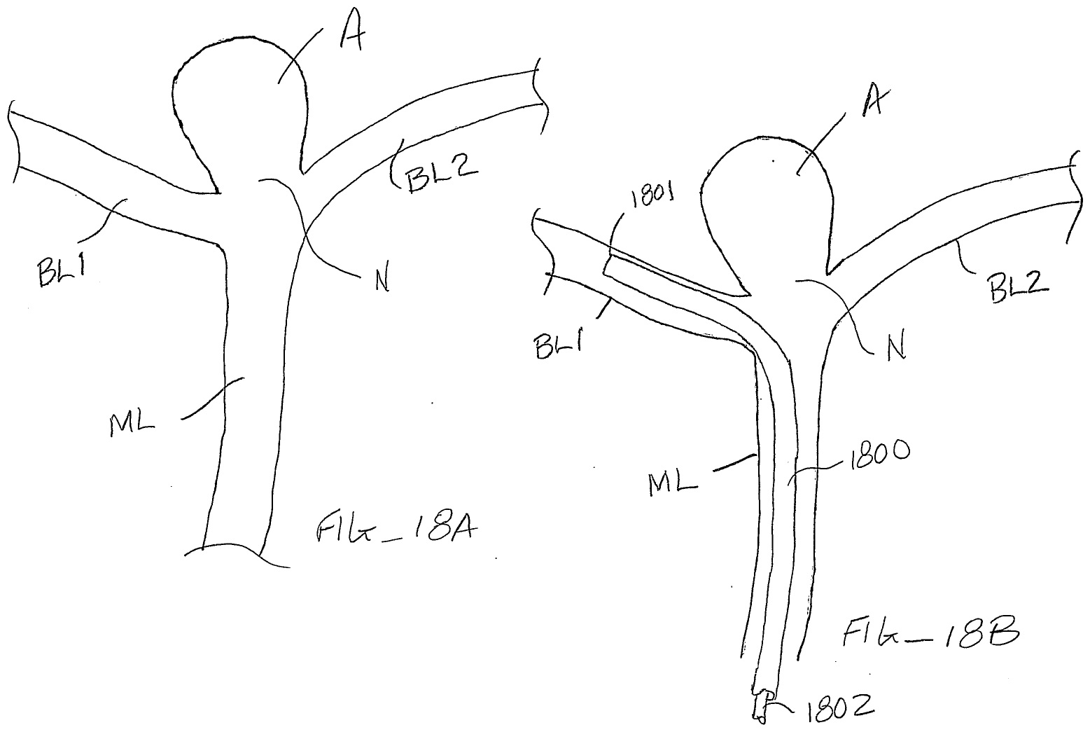

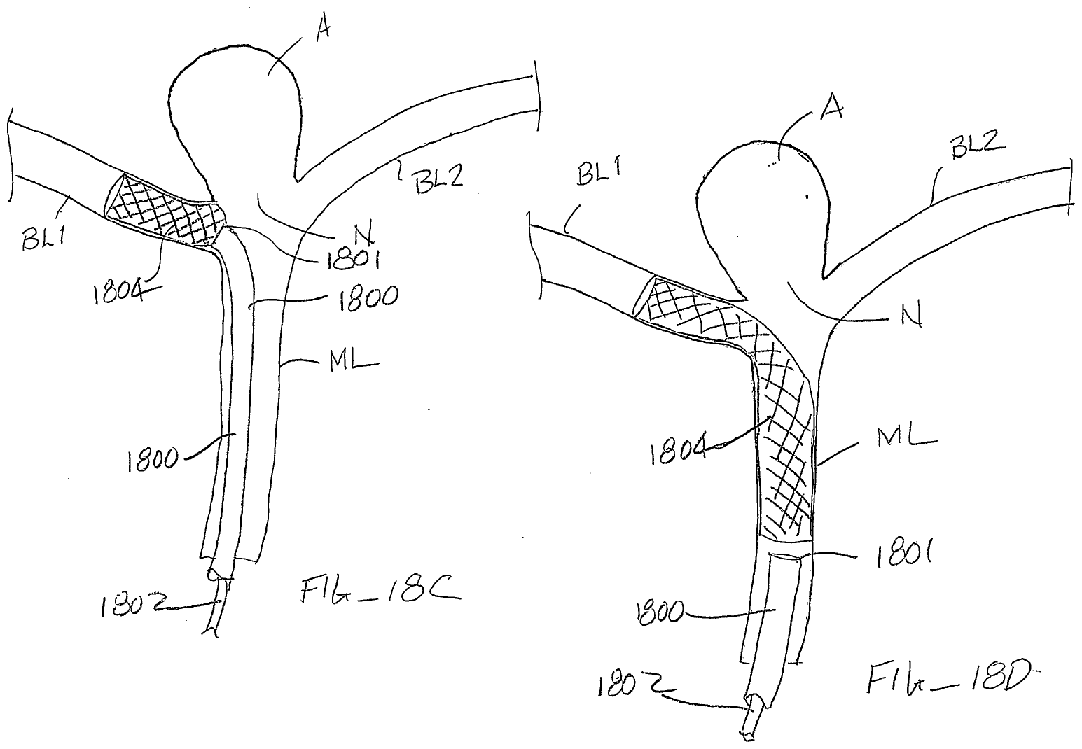

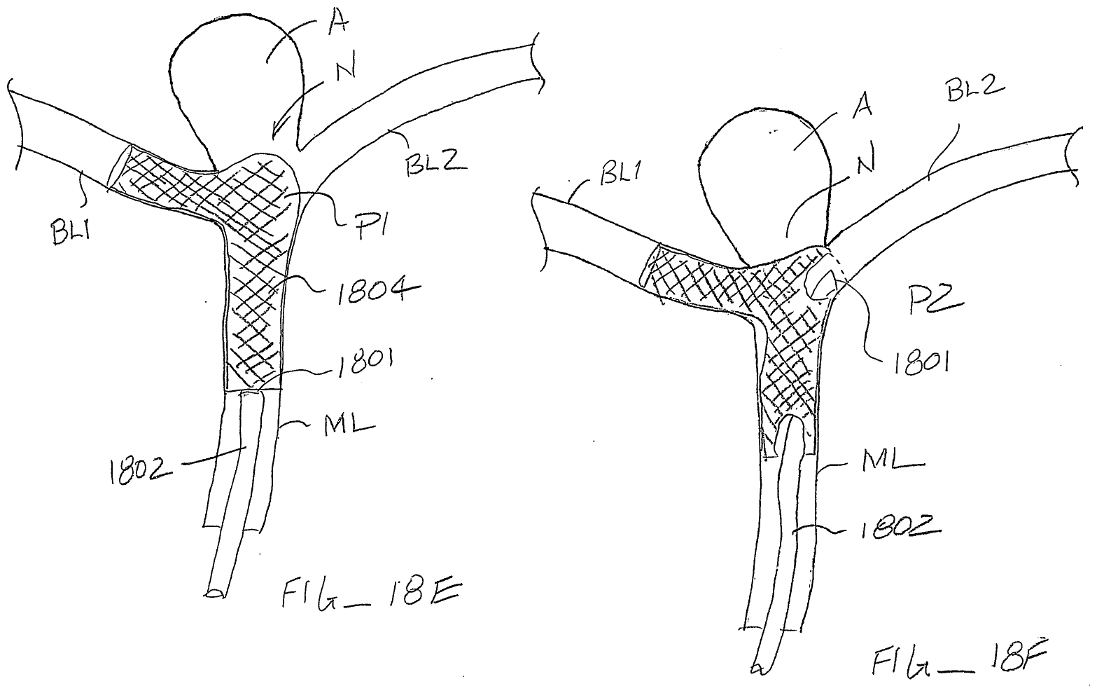

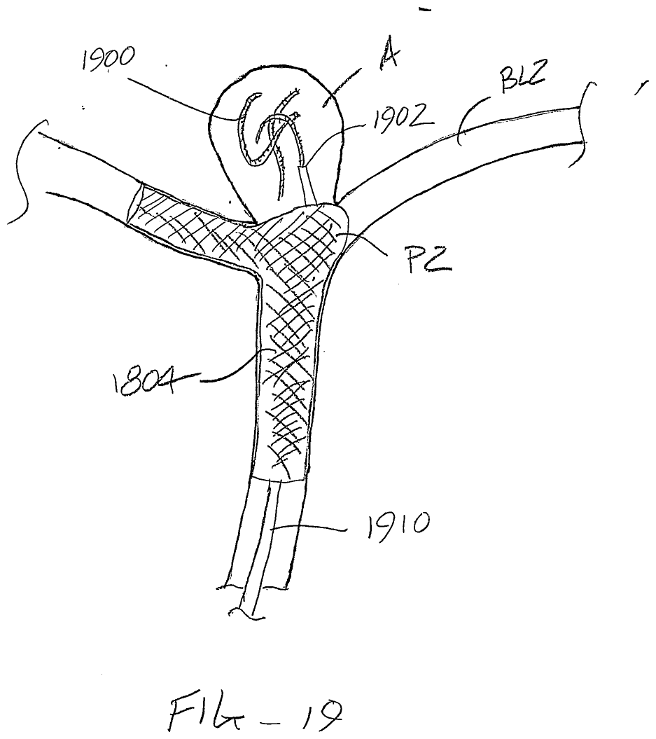

Referring now to FIGS. 18A to 18F, methods for delivering the flow diverting stents of the present invention to a cerebral aneurysm A will be described. While the specific description relates to stent delivery at an aneurysm A having a neck N located at a vascular bifurcation having a main lumen ML, a first branch lumen, and a second branch lumen BL2, the methods described will work as well with wide neck and other sidewall aneurysms. As shown in FIG. 18A, the aneurysm A is located at the base or “groin” of the bifurcation and separates the openings into each of the branch vessels BL1 and BL2.

A flow diverting stent 1804 is delivered to the aneurysm A by a microcatheter 1800 having a distal end 1801 which is introduced into the first branch lumen BL1 (although the tip might just as well have been introduced into the second branch lumen BL2) as shown in FIG. 18B. The self-expanding flow directing stent 1804 is constrained within the distal portion of the microcatheter 1800 during advancement through the vasculature.

After reaching the target delivery location, as shown in FIG. 18B, the tip 801 of the microcatheter 1800 is retracted proximally to release the stent 1804 from constraint, as shown in FIG. 18C, allowing the stent to expand into the first side branch SB1 and the main lumen ML. A pusher 1802 or similar structure engages a proximal end of the stent 1804 to hold the stent in place as the microcatheter is retracted.

As show in FIG. 18D, however, while stent 1804 can closely appress or engage the luminal walls of the main lumen ML and first side branch lumen SB1, the bifurcated geometry can limit or prevent coverage over the neck N of the aneurysm A, reducing the effectiveness of stent placement. In order to enhance coverage of the bifurcated aneurysm neck, it has been found that the preferred flow directing stents of the present invention can be “shaped” to partially or fully cover the neck, as shown in FIGS. 18E and 18F.

In particular, the resiliently deformable porous tubular bodies of the flow directed stents of the present invention have elastic and tensile properties which improve their elastic deformation and recovery to allow post-release shaping to cover the neck and/or portions of the opening into the second branch lumen SB2.

In specific examples, the metal wires may be formed from metals having a modulus of elasticity in a range from 5 GPa to 30 GPa and the bioabsorbable polymeric fibers may be formed from a polymer have a modulus of elasticity in a range from 2 GPa to 10 GPa.

In exemplary cases, the metal wires are formed from a nickel-titanium alloy and the bioabsorbable polymeric fibers are formed from poly-L-Lactic acid (PLLA). By comparison, the elastic moduli of metals and metal alloys typically used in stents, such as Co—Cr and Pt, are much higher, e.g., on the order of 210 GPa and 170 GPa, respectively, which makes their elastic deformation and recovery more difficult and also makes them more prone to kinking under high strain.

As show in FIGS. 18E and 18F, the stent 1804 configuration of FIG. 18D be modified to include a protrusion to extend partially (P1, FIG. 18E) or fully (P2, FIG. 18F) over the neck N of the aneurysm A. The modification can be effected by stressing or compressing the resiliently deformable porous tubular body using the microcatheter 1800, pusher 1802, or other elongate member introduced through the main lumen. For example, as shown in FIG. 18E, the distal tip 1801 of the microcatheter 1800 can be used to push against a proximal end of the flow directing stent 1804 after deployment to cause the protrusion P1 to project or “bulge” radially outwardly.

Alternatively or additionally, as shown in FIG. 18E, the distal tip 1801 of the microcatheter 1800 can be used to push an interior surface of the flow directing stent 1804 after deployment to push out the protruding region P2.

Referring now to FIG. 19, a particular advantage of the flow diverting stents of the present invention is the ability to deliver occlusive coils 1900 and/or other occluding materials (such as glues, fillers, clot inducing agents, and the like) through the wall of the resiliently deformable porous tubular body after deployment. A distal tip 1902 of a coil delivery catheter 1910, typically having an outside diameter in a range from 0.5 mm to 0.8 mm, is advanced through the braid of interwoven metal wires and bioabsorbable polymeric fibers to enter an interior of the aneurysm, and the occlusive coils delivered as illustrated. The polymeric fibers of the present invention are sufficiently elastic to allow both passage of the coil delivery catheter tip 1902 through the braid and to close upon removal of the tip, creating an effective barrier to retain the occlusive coils or other occluding material, While illustrated with the stent 1804 located at a vascular bifurcation, the ability to deliver coils and other occlusive materials through the walls of the flow diverting stents of the present invention applies equally to the stents implanted at sidewall aneurysms and all other vasculature locations.

While specific embodiments of the invention have been described and illustrated, such embodiments should be considered illustrative of the invention only and not as limiting the invention as construed in accordance with the accompanying claims.

What is claimed is:

1. A method for diverting blood flow in an intracranial blood vessel away from an aneurysm, the method comprising:
expanding a device comprising a resiliently deformable porous tubular body in a lumen of the intracranial blood vessel across a neck of the aneurysm, said resiliently deformable porous tubular body comprising metal wires interwoven with bioabsorbable polymeric fibers to form a braid, wherein the metal wires are resiliently deformable and configured to fully expand the tubular structure against the body wall;
wherein the braid has a porosity when in an expanded configuration selected to permit a small amount of blood to enter the aneurysm with low velocity which causes thrombosis and occlusion of the aneurysm and permits the aneurysm to heal; and
wherein the bioabsorbable polymeric fibers degrade over time after the aneurysm has healed leaving only the metal wires in place.

2. The method of claim 1, wherein the braid has a porosity in a range of about 60% to about 80% when the tubular body is expanded.

3. The method of claim 1, wherein the braid comprises (i) at least 38 bioabsorbable polymeric fibers and (ii) from 2 to 12 resiliently deformable interwoven metal wires.

4. The method of claim 1, wherein the bioabsorbable polymeric fibers have a diameter in the range of about 30 μm to about 80 μm.

5. The method of claim 1, wherein the metal wires are radiopaque and the method further comprises visualizing the radiopaque wires while the tubular body of the device is being deployed.

6. The method of claim 1, wherein the resiliently deformable wire comprises a nickel-titanium alloy or a cobalt-chromium-nickel alloy.

7. The method of claim 6, wherein the bioabsorbable polymeric fibers comprise one or more materials selected from the group consisting of polylactides (PLA), polylactide-co-glycolides (PLGA), DLPLA-poly(dl-lactide), poly-L-Lactic acid), LPLA-poly(l-lactide), PGA-LPLA-poly(l-lactide-co-glycolide), PGA-DLPLA-poly(dl-lactide-co-glycolide), LPLA-DLPLA-poly(l-lactide-co-dl-lactide), or any combination thereof.

8. The method of claim 1, further comprising delivering an occlusive material to the aneurysm after the porous tubular body has been expanded across the neck of the aneurysm.

9. The method of claim 8, wherein delivering the occlusive material to the aneurysm after the porous tubular body has been expanded across the neck of the aneurysm comprises advancing a distal tip of a microcatheter through the braid and delivering the occlusive material through the microcatheter.

10. The method of claim 9, wherein the microcatheter is advanced through the polymeric fibers causing the braid to expand over the microcatheter when introduced and to collapse when to microcatheter is withdrawn.

11. The method of claim 9, wherein the occlusive material comprises occluding coils.

12. The method of claim 1, wherein the metal wires are formed from a metal have a modulus of elasticity in a range from 5 GPa to 30 GPa and the bioabsorbable polymeric fibers are formed from a polymer have a modulus of elasticity in a range from 2 GPa to 10 GPa.

13. The method of claim 12, wherein the metal wires are formed from a nickel-titanium alloy and the bioabsorbable polymeric fibers are formed from PLLA.

14. The method of claim 1, wherein the resiliently deformable porous tubular body is shaped across and/or into at least the neck of the aneurysm.

15. The method of claim 20, the resiliently deformable porous tubular body is expanded across a neck of a sidewall aneurysm.

16. The method of claim 20, the resiliently deformable porous tubular body is expanded across and/or into (a) the neck of a bifurcated aneurysm and/or (b) the neck of a branch lumen.

17. A method for diverting blood flow in an intracranial blood vessel away from an aneurysm located at a bifurcation, the method comprising:
expanding a device comprising a resiliently deformable porous tubular body from a lumen of the intracranial blood vessel into one of the two branch lumens, said resiliently deformable porous tubular body comprising metal wires interwoven with bioabsorbable polymeric fibers to form a braid, wherein the metal wires are resiliently deformable and configured to fully expand the tubular structure against the body wall; and
shaping the resiliently deformable porous tubular body across and/or into at least one of (i) a neck of the aneurysm and (ii) the other of the two branch lumens after the resiliently deformable porous tubular body has been expanded;
wherein the braid has a porosity when in an expanded configuration selected to permit a small amount of blood to enter the aneurysm with low velocity which causes thrombosis and occlusion of the aneurysm and permits the aneurysm to heal; and
wherein the bioabsorbable polymeric fibers degrade over time leaving only the metal wires in place.

18. The method of claim 17, wherein the resiliently deformable porous tubular body is shaped across and/or into at least the neck of the aneurysm.

19. The method of claim 17, the resiliently deformable porous tubular body is shaped across and/or into at least the other of the two branch lumens.

20. The method of claim 17, the resiliently deformable porous tubular body is shaped across and/or into both (i) the neck of the aneurysm and (ii) the other of the two branch lumens.

21. The method of claim 17, wherein expanding the device comprises releasing the device from constraint within a microcatheter and shaping the resiliently deformable porous tubular body comprises pushing on the resiliently deformable porous tubular body with the microcatheter and/or a pusher member advanced through the microcatheter.

22. The method of claim 17, wherein the metal wires are formed from a metal have a modulus of elasticity in a range from 5 GPa to 30 GPa and the bioabsorbable polymeric fibers are formed from a polymer have a modulus of elasticity in a range from 2 GPa to 10 GPa.

23. The method of claim 17, wherein the metal wires are formed from a nickel-titanium alloy and the bioabsorbable polymeric fibers are formed from PLLA.
